(19) United States
(12) Patent Application Publication
(10) Pub. No.: US 2003/0232365 A1
(43) Pub. Date: Dec. 18, 2003

(54) BDNF polymorphisms and association with bipolar disorder

(75) Inventors: Pamela Sklar, Brookline, MA (US); Eric S. Lander, Cambridge, MA (US); Melvin G. McInnis, Timonium, MD (US); J. Raymond DePaulo JR., Baltimore, MD (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignees: Whitehead Institute for Biomedical Research, Cambridge, MA; Johns Hopkins University, Baltimore, MD; The General Hospital Corporation, Boston, MA

(21) Appl. No.: 10/409,041

(22) Filed: Apr. 7, 2003

Related U.S. Application Data
(63) Continuation-in-part of application No. 10/077,171, filed on Feb. 15, 2002.
(60) Provisional application No. 60/371,260, filed on Apr. 8, 2002.
(60) Provisional application No. 60/269,059, filed on Feb. 15, 2001.

(51) Int. Cl. C12Q 1/68

(52) U.S. Cl. 435/6

(57) ABSTRACT

Methods for diagnosing and treating neuropsychiatric disorders, especially bipolar disorder, and to methods for identifying compounds for use in the diagnosis and treatment of neuropsychiatric disorders are disclosed. Also disclosed are novel compounds and pharmaceutical compositions for use in the diagnosis and treatment of neuropsychiatric disorders such as bipolar disorder.

### RELATED APPLICATIONS

This application claims the benefit of U.S. Provisional Application No. 60/371,260, filed on Apr. 8, 2002. This application is also a continuation-in-part of U.S. application Ser. No. 10/077,171, filed Feb. 15, 2002, which claims the benefit of U.S. Provisional Application No. 60/269,059, filed on Feb. 15, 2001. The entire teachings of the above applications are incorporated herein by reference.

### BACKGROUND OF THE INVENTION

The human genome is made of about 3 billion pairs of bases (adenine (A), cytosine (C), thymine (T) and guanine (G)). In one out of every 1,000 of these pairs, one individual has e.g., an A, where another individual has, e.g., a T. These single letter DNA differences, called single nucleotide polymorphisms (SNPs), between individuals comprise most genetic variation and thus underlie disease susceptibility. An effort is underway to identify and interpret individual variations.

Haplotypes are ancestral segments of chromosomes that contain many single letter genetic variations inherited together as a set or a block, and they can be used to decipher the genetic differences that make some people more susceptible to disease than others. Due to the size of the human genome, identifying a haplotype would make finding disease genes a manageable task. Instead of searching through a giant haystack of millions of SNPs, one could search through bundles of 10,000 to 50,000 bases each. One goal of modern science is to identify specific haplotypes and determine which haplotype(s) is associated with a specific phenotype.

Modern psychiatry typically subdivides mood disorders into bipolar disorders (episodes of mania or both mania and depression) and unipolar depressive disorder (episodes of depression). Symptoms of mania include expansive, elevated or irritable mood, inflated self-esteem, grandiosity, decreased need for sleep, increased talkativeness, racing thoughts, distractibility, increased goal-directed activity, and excessive involvement in pleasurable activities with a high potential for painful consequences. Depressive symptoms include depressed mood, diminished interest or pleasure in activities, insomnia or hypersomnia, psychomotor agitation or retardation, fatigue or loss of energy, feelings of worthlessness, excessive guilt, inability to concentrate or act decisively, and recurrent thoughts of death or suicide. Several mental disorders have been proposed as alternate expressions of a bipolar disorder, including schizoaffective disorder, recurrent unipolar depression and hypomania (bipolar II, disorder).

Neuropsychiatric disorders, such as schizophrenia, attention deficit disorders, schizoaffective disorders, bipolar disorders and unipolar disorders, differ from neurological disorders in that anatomical or biochemical pathologies are readily detectable for the latter but not the former. Largely as a result of this difference, drugs which have been used to treat individuals with neuropsychiatric disorders, including lithium salts, valproic acid and carbamazepine, have not been predictably effective in treatment regimens across a variety of patients. Treatment regimens are further complicated by the fact that clinical diagnosis currently relies on clinical observation and subjective reports. Identification of the anatomical or biochemical defects which result in neuropsychiatric disorders is needed in order to effectively distinguish between the disorders and to allow the design and administration of effective therapeutic agents and treatment regimens for these disorders. Identification of individual SNPs and/or haplotypes which are associated with neuropsychiatric disorders will aid in the diagnosis and treatment of these disorders.

### SUMMARY OF THE INVENTION

Six haplotypes formed by alleles of eight single nucleotide polymorphisms (SNPs) in or near the coding region of the BDNF gene have been discovered. One of the haplotypes is transmitted significantly more often from parents to children with bipolar disorder than would be expected by chance, while another is transmitted significantly less often than would be expected by chance. Thus, the invention relates to the SNPs identified as described herein, both singly and in combination, as well as to the use of these SNPs, and other SNPs nearby in linkage disequilibrium with these SNPs, for diagnosis, prediction of clinical course and treatment response for neuropsychiatric disorders, development of new treatments for neuropsychiatric disorders based upon comparison of the variant and normal (reference) versions of the gene or gene product, and development of cell culture-based and animal models for research and treatment of neuropsychiatric disorders. The invention further relates to novel compounds and pharmaceutical compositions for use in the diagnosis and treatment of such disorders.

In one embodiment the BDNF gene has the nucleotide sequence of SEQ ID NO: 1. In a further embodiment, the invention relates to a method for predicting the likelihood that an individual will have a neuropsychiatric disorder, comprising the steps of obtaining a nucleic acid sample from an individual to be assessed and determining the nucleotide present at nucleotide position 11,757 relative to the start codon of the brain-derived neurotrophic factor (BDNF) gene, wherein the presence of a cytosine at position 11,757 indicates that the individual has a greater likelihood of having a neuropsychiatric disorder than an individual having a guanine at that position. In a particular embodiment, the neuropsychiatric disorder is bipolar disorder and/or the individual is an individual at risk for development of bipolar disorder.

In another embodiment, the invention relates to a method for predicting the likelihood that an individual will have a neuropsychiatric disorder, comprising the steps of obtaining a nucleic acid sample from an individual to be assessed and determining the nucleotide present at nucleotide position 14,569 relative to the start codon of the brain-derived neurotrophic factor (BDNF) gene, wherein the presence of a guanine at position 14,569 indicates that the individual has a greater likelihood of having a neuropsychiatric disorder than an individual having an adenine at that position. In a particular embodiment, the neuropsychiatric disorder is bipolar disorder and/or the individual is an individual at risk for development of bipolar disorder.

In another embodiment, the invention relates to a method for predicting the likelihood that an individual will have reduced symptomology associated with a neuropsychiatric disorder, comprising the steps of obtaining a nucleic acid sample from an individual to be assessed and determining the nucleotide present at nucleotide position 11,757 relative to the start codon of the brain-derived neurotrophic factor (BDNF) gene, wherein the presence of a guanine at position 11,757 indicates that the individual has a greater likelihood of having reduced symptomology associated with a neuropsychiatric disorder than an individual having a cytosine at that position. In a particular embodiment, the neuropsychiatric disorder is bipolar disorder.

In another embodiment, the invention relates to a method for predicting the likelihood that an individual will have reduced symptomology associated with a neuropsychiatric disorder, comprising the steps of obtaining a nucleic acid sample from an individual to be assessed and determining the nucleotide present at nucleotide position 14,569 relative to the start codon of the brain-derived neurotrophic factor (BDNF) gene, wherein the presence of an adenine at position 14,569 indicates that the individual has a greater likelihood of having reduced symptomology associated with a neuropsychiatric disorder than an individual having a guanine at that position. In a particular embodiment, the neuropsychiatric disorder is bipolar disorder.

In another embodiment, the invention relates to a method of diagnosing or aiding in the diagnosis of a neuropsychiatric disorder in an individual comprising obtaining a nucleic acid sample from the individual and determining the nucleotide present at two or more of nucleotide positions −633, 196, 11757 and 14569 relative to the start codon of the brain-derived neurotrophic factor (BDNF) gene, wherein presence of one or more of an adenine at nucleotide position −633, a guanine at nucleotide position 196, a cytosine at nucleotide position 11757 or a guanine at nucleotide position 14569 is indicative of increased likelihood of a neuropsychiatric disorder in the individual as compared with an individual having one or more of a thymine at nucleotide position −633, an adenine at nucleotide position 196, a guanine at nucleotide position 11757 or an adenine at nucleotide position 14569. In a particular embodiment, the neuropsychiatric disorder is bipolar disorder. In a preferred embodiment, the nucleotide present at all four of nucleotide positions −633, 196, 11757 and 14569 relative to the start codon of the brain-derived neurotrophic factor (BDNF) gene is determined.

In another embodiment, the invention relates to a method of diagnosing or aiding in the diagnosis of a neuropsychiatric disorder in an individual comprising obtaining a nucleic acid sample from the individual and determining the nucleotide present at two or more of nucleotide positions −633, 196, 11757 and 14569 relative to the start codon of the brain-derived neurotrophic factor (BDNF) gene, wherein presence of one or more of a thymine at nucleotide position −633, an adenine at nucleotide position 196, a guanine at nucleotide position 11757 or an adenine at nucleotide position 14569 is indicative of decreased likelihood of a neuropsychiatric disorder in the individual as compared with an individual having one or more of an adenine at nucleotide position −633, a guanine at nucleotide position 196, a cytosine at nucleotide position 11757 or a guanine at nucleotide position 14569. In a particular embodiment, the neuropsychiatric disorder is bipolar disorder. In a preferred embodiment, the nucleotide present at all four of nucleotide positions −633, 196, 11757 and 14569 relative to the start codon of the brain-derived neurotrophic factor (BDNF) gene is determined.

In another embodiment, the invention relates to a method for predicting the likelihood that an individual will have a neuropsychiatric disorder, comprising the steps of obtaining a nucleic acid sample from an individual to be assessed and determining the nucleotide present at two or more of nucleotide positions −633, 196, 11757 and 14569 relative to the start codon of the brain-derived neurotrophic factor (BDNF) gene, wherein presence of one or more of an adenine at nucleotide position −633, a guanine at nucleotide position 196, a cytosine at nucleotide position 11757 or a guanine at nucleotide position 14569 is indicative of increased likelihood of a neuropsychiatric disorder in the individual as compared with an individual having one or more of a thymine at nucleotide position −633, an adenine at nucleotide position 196, a guanine at nucleotide position 11757 or an adenine at nucleotide position 14569. In a particular embodiment, the neuropsychiatric disorder is bipolar disorder. In a preferred embodiment, the nucleotide present at all four of nucleotide positions −633, 196, 11757 and 14569 relative to the start codon of the brain-derived neurotrophic factor (BDNF) gene is determined. In a particular embodiment, the neuropsychiatric disorder is bipolar disorder and/or the individual is an individual at risk for development of a neuropsychiatric disorder.

In another embodiment, the invention relates to a method for predicting the likelihood that an individual will have a neuropsychiatric disorder, or for aiding in the diagnosis of a neuropsychiatric disorder, or predicting the likelihood of having symptomology associated with a neuropsychiatric disorder, comprising the steps of obtaining a nucleic acid sample from an individual to be assessed and determining the nucleotide present at one or more of the nucleotide positions disclosed herein relative to the start codon of the BDNF gene. In a preferred embodiment, the nucleotides present at one or more of these nucleotide positions are determined, in a particularly preferred embodiment the nucleotides present at two or more of these nucleotide positions are determined, in a more particularly preferred embodiment the nucleotides present at three or more of these nucleotide positions are determined, in an even more particularly preferred embodiment the nucleotides present at four or more, five or more, six or more, seven or more, or eight of these nucleotide positions are determined.

In another embodiment, the invention relates to a method for predicting the likelihood of the presence of one or more nucleotides comprising a haplotype associated with a neuropsychiatric disorder, comprising the steps of obtaining a nucleic acid sample from an individual to be assessed and determining the nucleotide present at one or more of the nucleotide positions disclosed herein relative to the start codon of the BDNF gene. In a preferred embodiment, the nucleotides present at one or more of these nucleotide positions are determined, in a particularly preferred embodiment the nucleotides present at two or more of these nucleotide positions are determined, in a more particularly preferred embodiment the nucleotides present at three or more of these nucleotide positions are determined, in an even more particularly preferred embodiment the nucleotides present at four or more, five or more, six or more, seven or more, or eight of these nucleotide positions are determined.

In a further embodiment, the invention relates to a nucleic acid molecule comprising the nucleic acid sequence of SEQ ID NO: 2, wherein the nucleic acid sequence comprises a polymorphic site at nucleotide positions −1480, −633, 196, 3071, 9202, 11757, 12910 and 14569 relative to the start codon for the brain-derived neurotrophic factor (BDNF) gene and wherein the nucleotide at one or more of the polymorphic sites is different from a nucleotide at a corresponding position in a corresponding reference allele. In a preferred embodiment, the nucleotide at one or more of the polymorphic sites is the variant nucleotide as shown in FIGS. 2A-2L.

In a further embodiment, the invention relates to a nucleic acid molecule comprising a portion of the nucleic acid sequence of SEQ ID NO: 2, which is at least ten nucleotides in length and which comprises at least one of the polymorphic sites selected from the group consisting of nucleotide positions −1480, −633, 196, 3071, 9202, 11757, 12910 and 14569.

In another embodiment, the invention is related to an oligonucleotide microarray having immobilized thereon a plurality of probes, wherein at least one of said probes is an allele-specific oligonucleotide which hybridizes specifically to a nucleic acid molecule comprising at least 10 contiguous nucleotides of the nucleic acid sequence of SEQ ID NO: 2 and comprising at least one of the polymorphic sites at nucleotide positions −1480, −633, 196, 3071, 9202, 11757, 12910 and 14569 relative to the start codon. As used herein, an “allele-specific oligonucleotide” is one which hybridizes specifically to one polymorphic form of a nucleotide sequence and not to other forms. Such allele-specific oligonucleotides can be used to distinguish between polymorphic forms.

### BRIEF DESCRIPTION OF THE DRAWINGS

FIGS. 1A through 1L show the polynucleotide sequence of 29 kb of genomic sequence of the region of chromosome 11 containing the BDNF gene (SEQ ID NO: 1).

FIGS. 2A through 2L show the location of SNPs that have been identified as described herein. 29 kb of genomic sequence in reverse complementarity relative to BDNF is shown (SEQ ID NO: 2). The box represents the start codon for the BDNF coding region. Positions of SNPs are indicated and their distance from the start codon for BDNF is assigned with regard to the 3′to 5′ orientation of BDNF. The “n's” represent unknown bases and are indicated to aid in avoidance of primers which span these regions.

FIG. 3 shows the results of genotyping of 8 BDNF SNPs in family-based samples.

FIG. 4 shows estimated haplotype probabilities and chi-squared test of multimarker haplotypes using TRANSMIT.

FIG. 5 is a schematic depicting the location of BDNF SNPs. Seventeen kb of genomic sequence are shown. The box represents the BDNF coding region. The arrow marks the amino terminus of the mature BDNF peptide. Positions of SNPs are indicated. Note that while a39 is not in the mature peptide, it is in a region highly conserved across species.

FIG. 6 shows the association between the G allele (BDNF SNP a39) and bipolar disorder for all three samples.

### DETAILED DESCRIPTION OF THE INVENTION

The development and maintenance of the vertebrate nervous system depends, in part, on the physiological availability of neuronal survival proteins known as neurotrophic factors. Neurotrophic factors play a role in maintaining neurons and their differentiated phenotypes in the adult nervous system. Nerve growth factor (NGF) remains the best characterized neurotrophic factor. However, brain-derived neurotrophic factor (BDNF) has been cloned and shown to be homologous to NGF (Leibrock et al., Nature 341:149-152 (1989); Hofer et al., EMBO J. 9:2459-2464 (1990); Maisonpierre et al., Genomics 10:558-568 (1991)). BDNF is initially synthesized as a 251 amino acid protein precursor that is subsequently cleaved to yield the mature protein. The mature form of BDNF essentially corresponds to the C-terminal half of its precursor and comprises 119 amino acids.

Work described herein relates to the discovery of six haplotypes formed by alleles of eight single nucleotide polymorphisms (SNPs) near or within the coding region of the BDNF gene. One of the haplotypes is transmitted significantly more often (overtransmitted) from heterozygous parents to children with bipolar disorder than would be expected by chance, while the other is transmitted less often (undertransmitted) than would be expected by chance. As used herein, “polymorphism” refers to the occurrence of two or more genetically determined alternative sequences or alleles in a population. A polymorphic marker or site is the locus at which divergence occurs. A polymorphic locus may be as small as one base pair, in which case it is referred to as a single nucleotide polymorphism (SNP). Furthermore, two or more polymorphic markers can be inherited together to form a haplotype.

As used herein, a predictive marker (SNP) is a polymorphic marker that is correlated with either an increased or reduced incidence of a phenotype (e.g., bipolar disorder). As used herein, an uncertain marker (SNP) is a polymorphic marker which is not correlated with either an increased or reduced incidence of a phenotype. One or more uncertain markers and one or more predictive markers, and various combinations thereof, can be inherited together to form a haplotype.

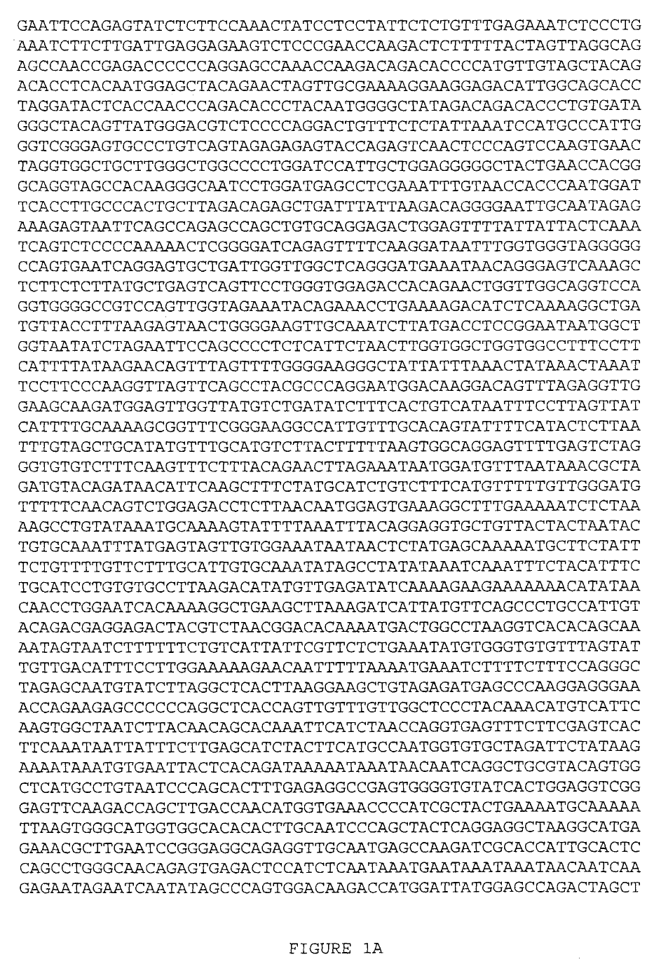

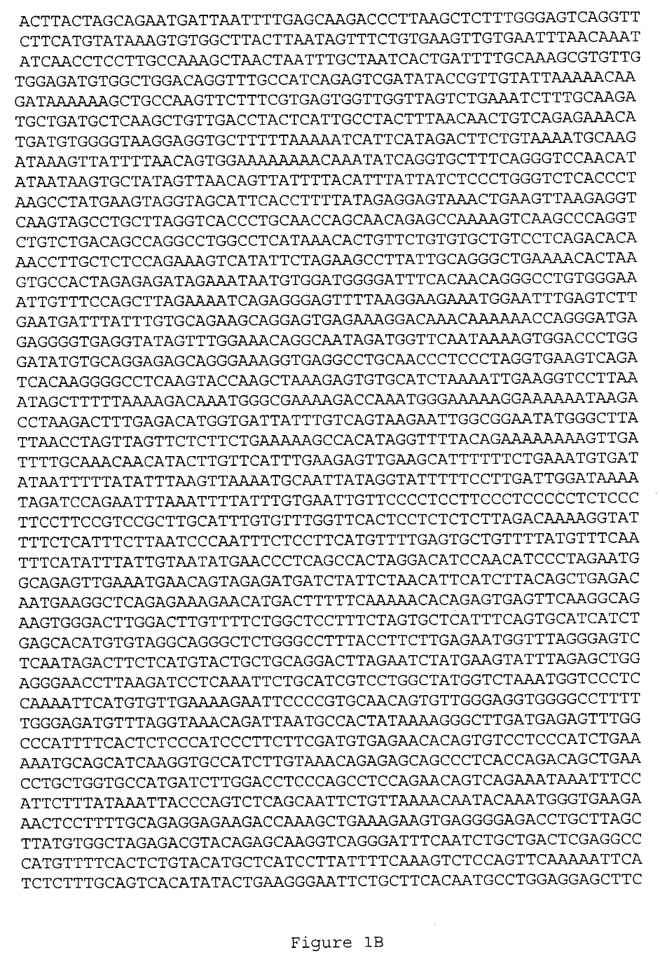

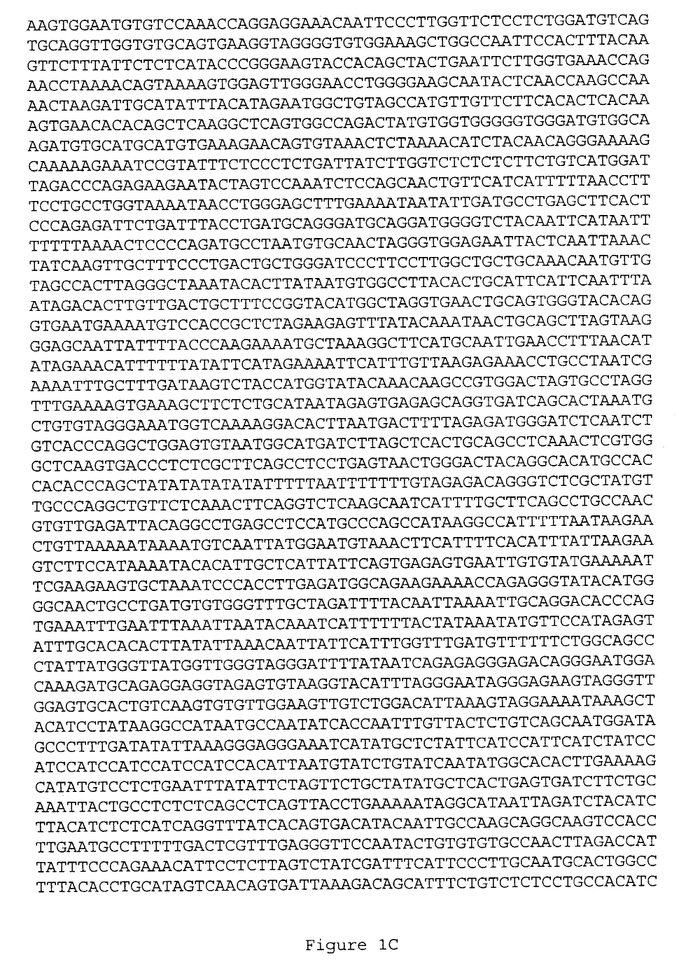

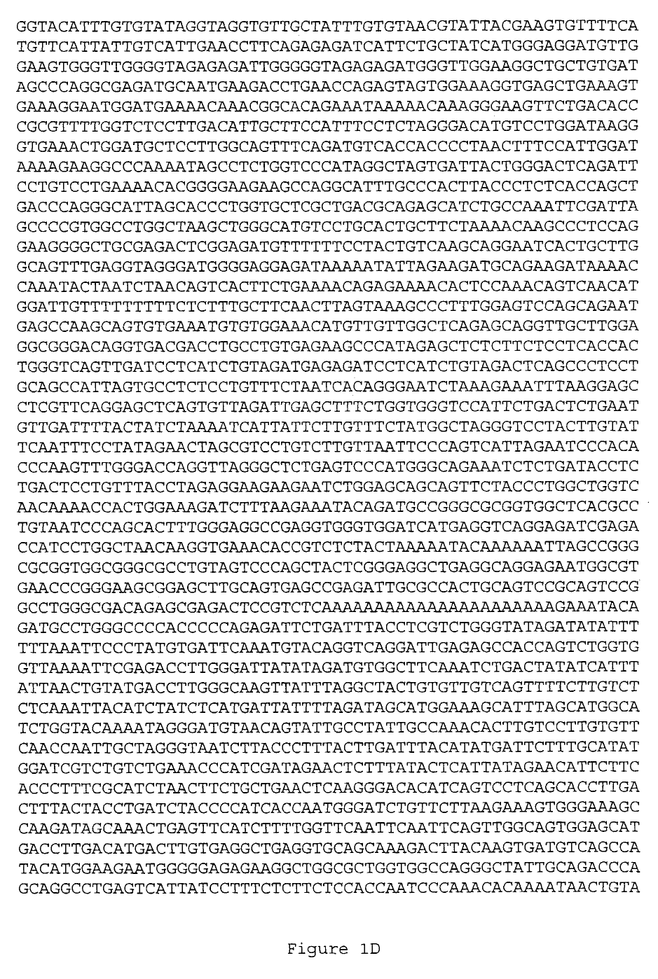

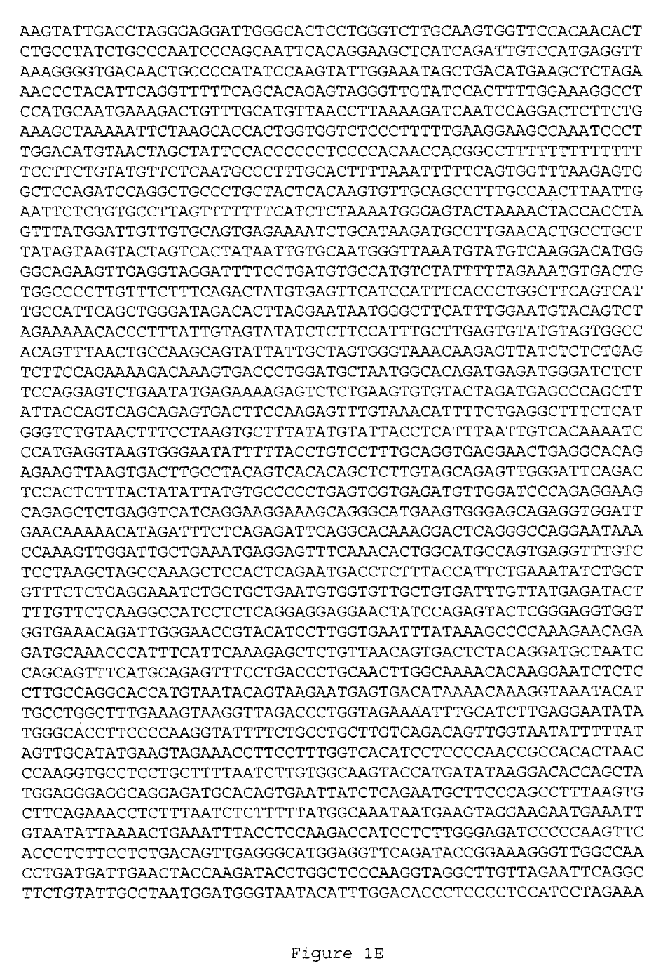

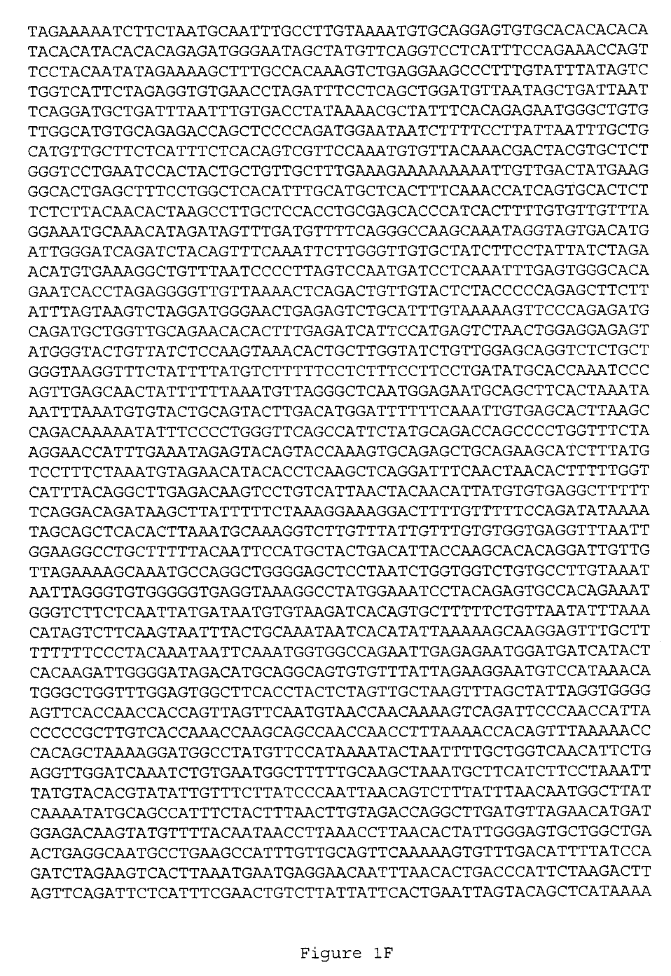

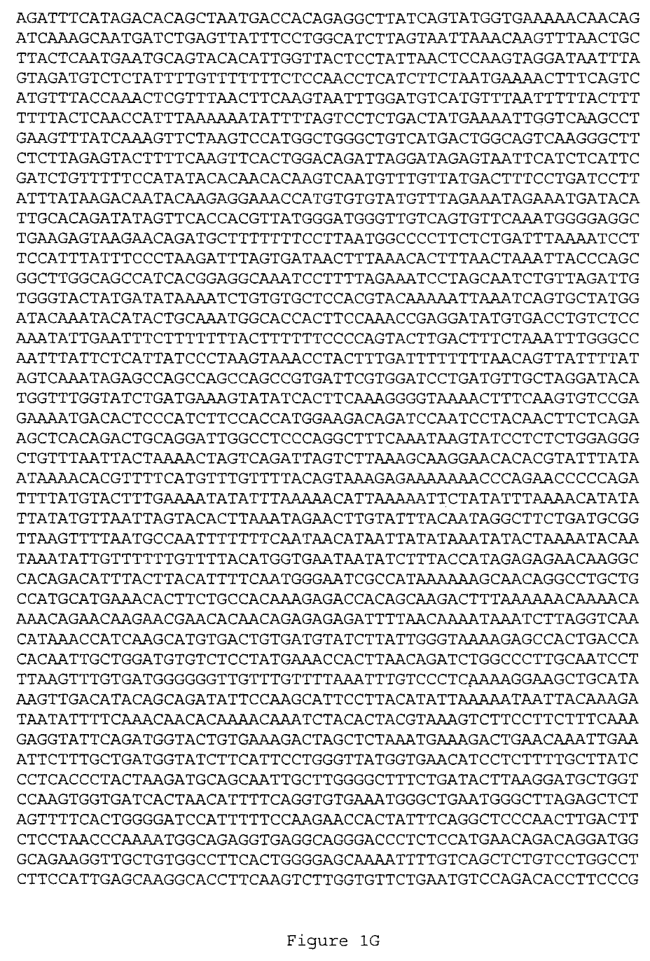

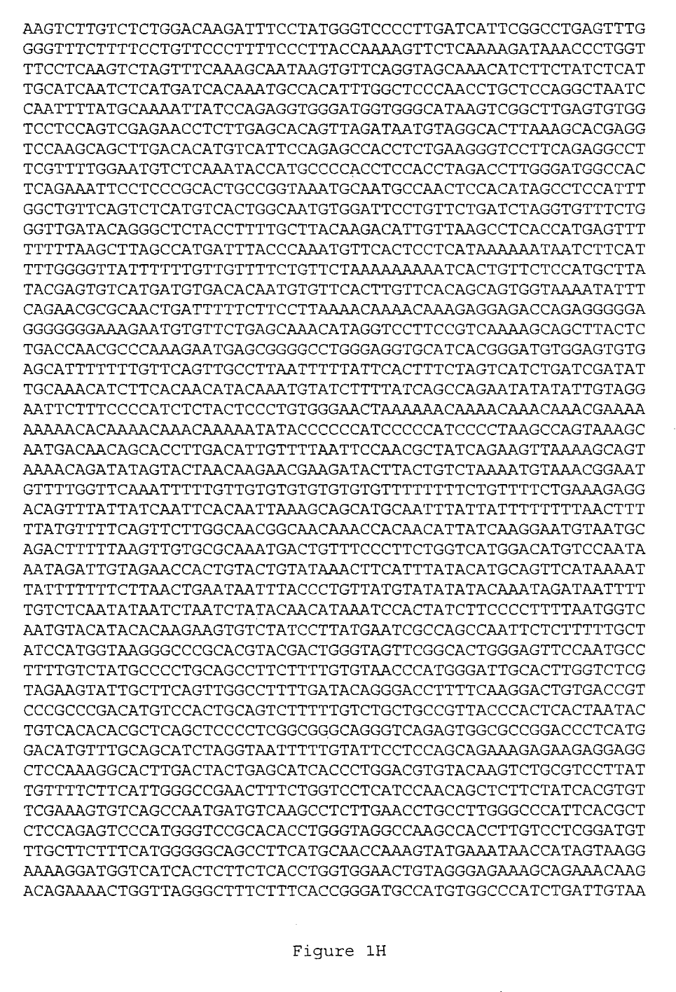

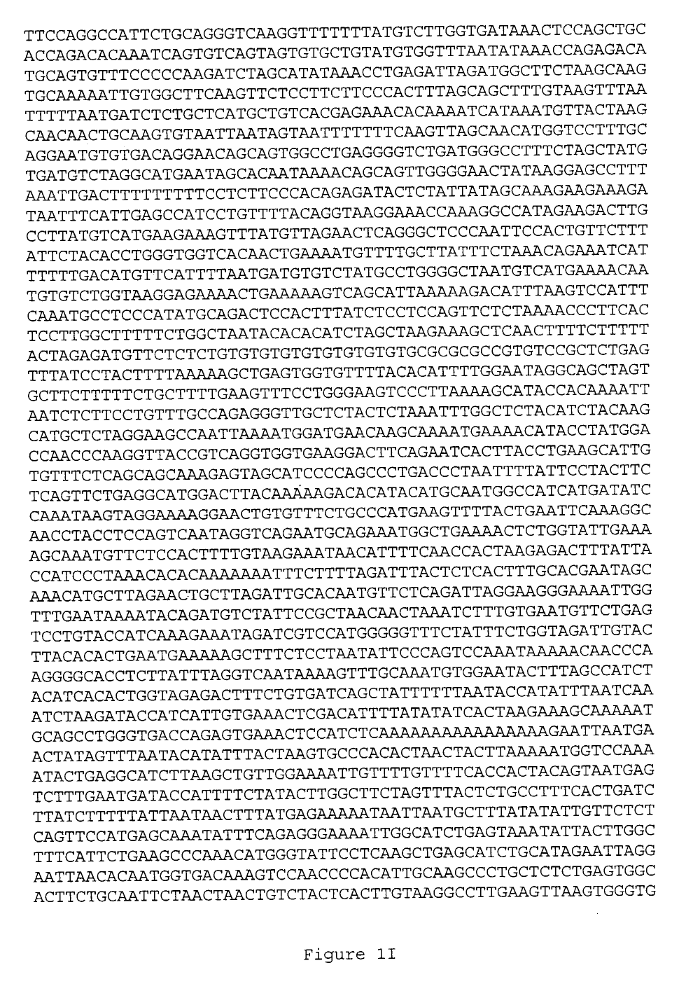

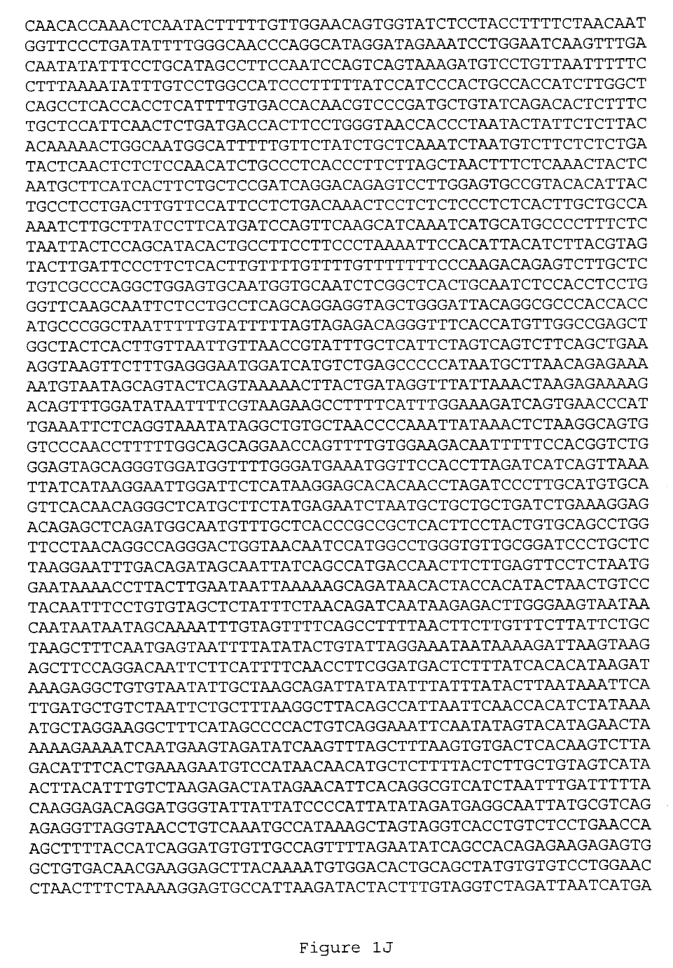

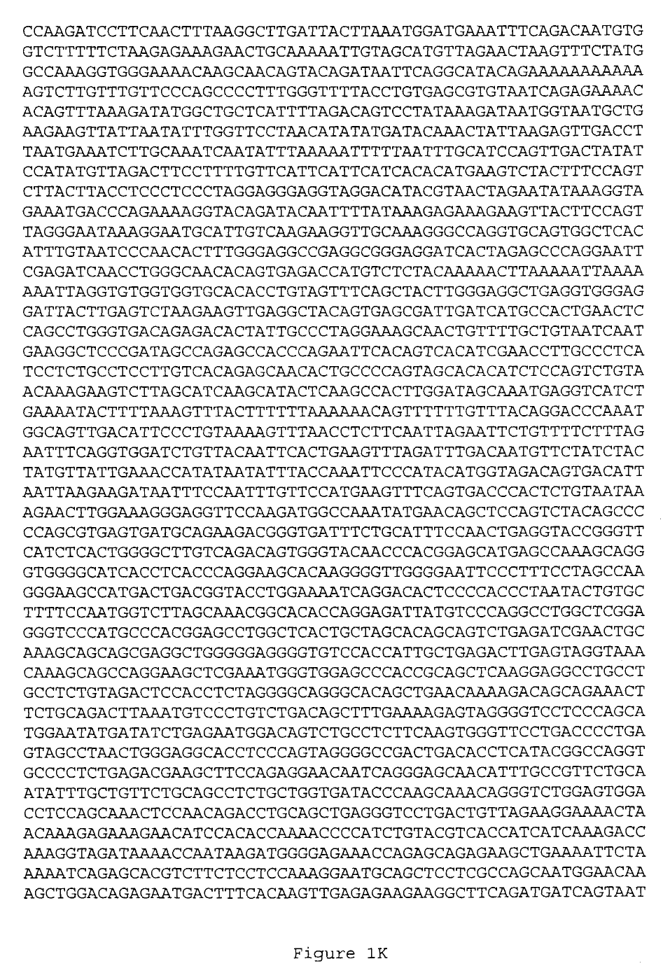

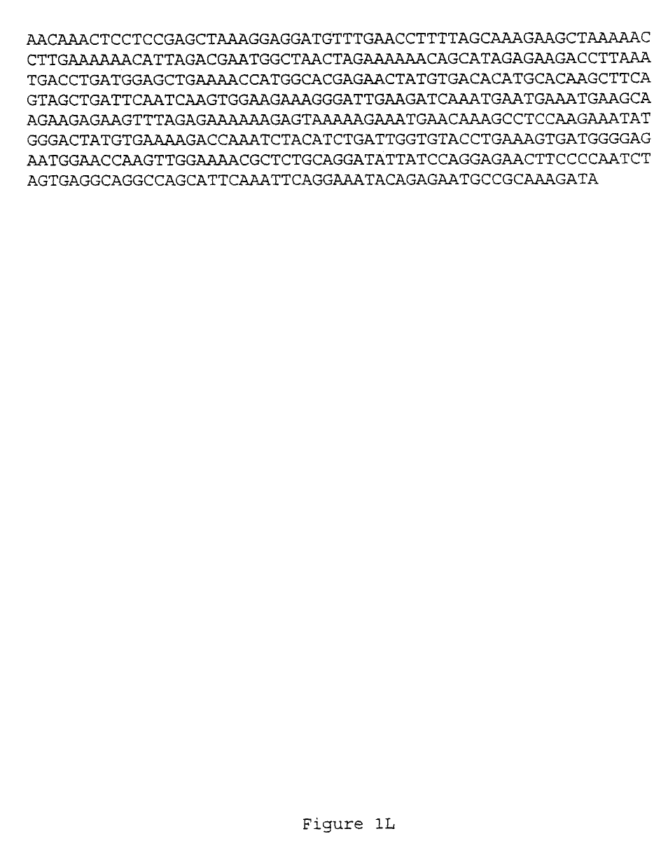

The initial genotyping strategy used to genotype SNPs within BDNF is described in the Examples. In order to identify haplotypes with significant transmission disequilibrium to map and identify a true susceptibility allele, additional SNPs were obtained for genotyping. Approximately 29 kilobases of contiguous genomic sequence (FIG. 1, SEQ ID NO: 1) found in the human genome browser was examined, including approximately 8 kb of 5′UTR. This was resequenced in DNA from 6 patients affected with bipolar I (BP1) disorder and 2 control DNAs.

44 SNPs were identified by this genotyping strategy using three test samples. One test sample comprised a population of 136 trios (Hopkins sample), where the proband for 109 trios has BP1 or SAB disorder (80%) and 27 trios where the proband has bipolar II (BPII) (20%). Another sample of 189 trios was obtained from the NIMH Genetics Initiative (NIMH sample), and a third sample of 145 trios (UK sample) was obtained from the UK collaboration between the University of Wales College of Medicine in Cardiff and the University of Birmingham in Birmingham.

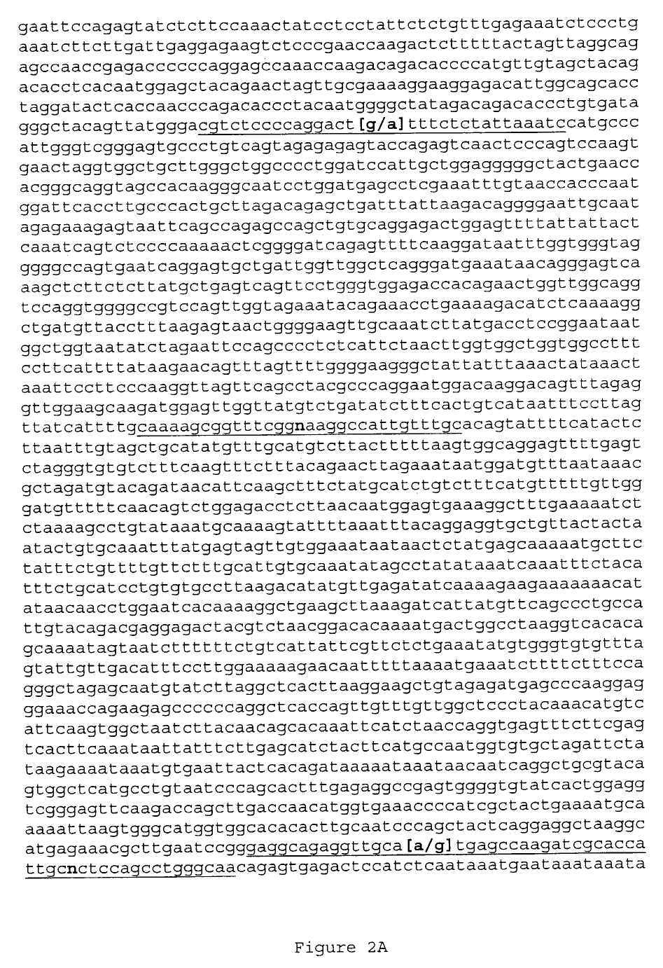

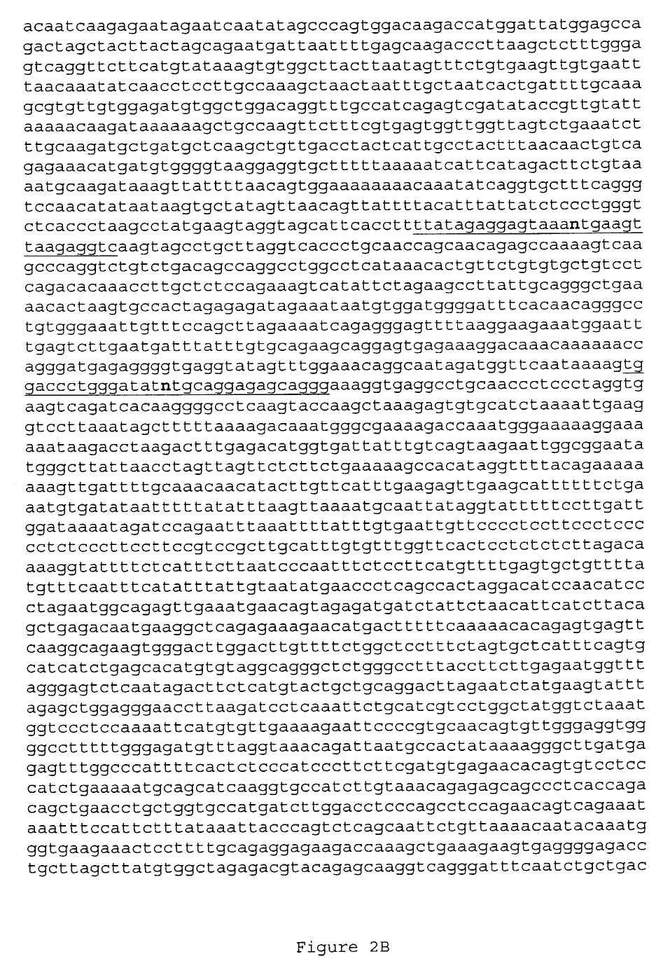

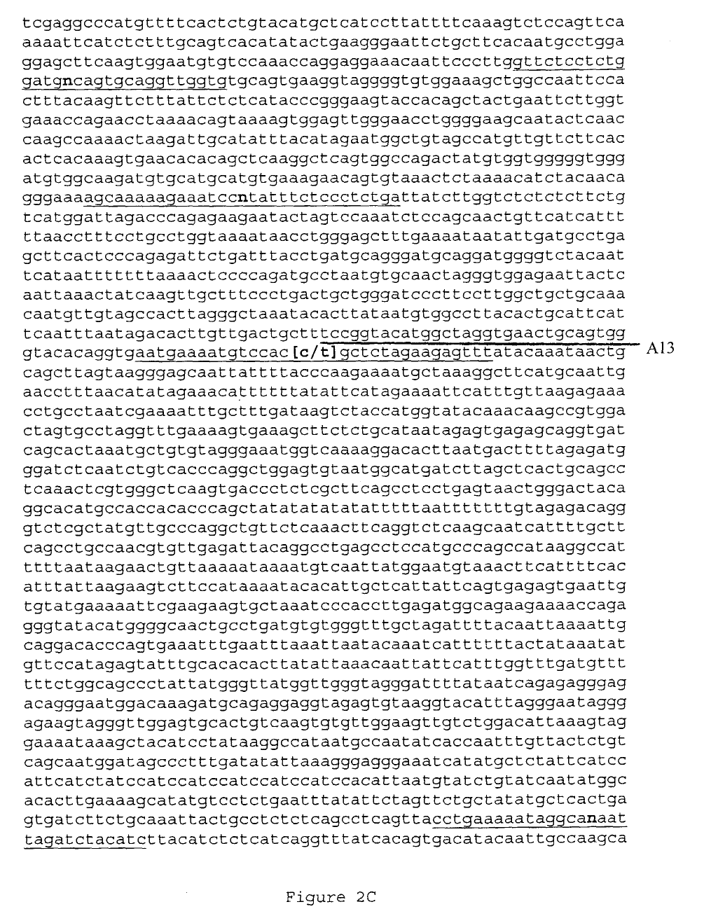

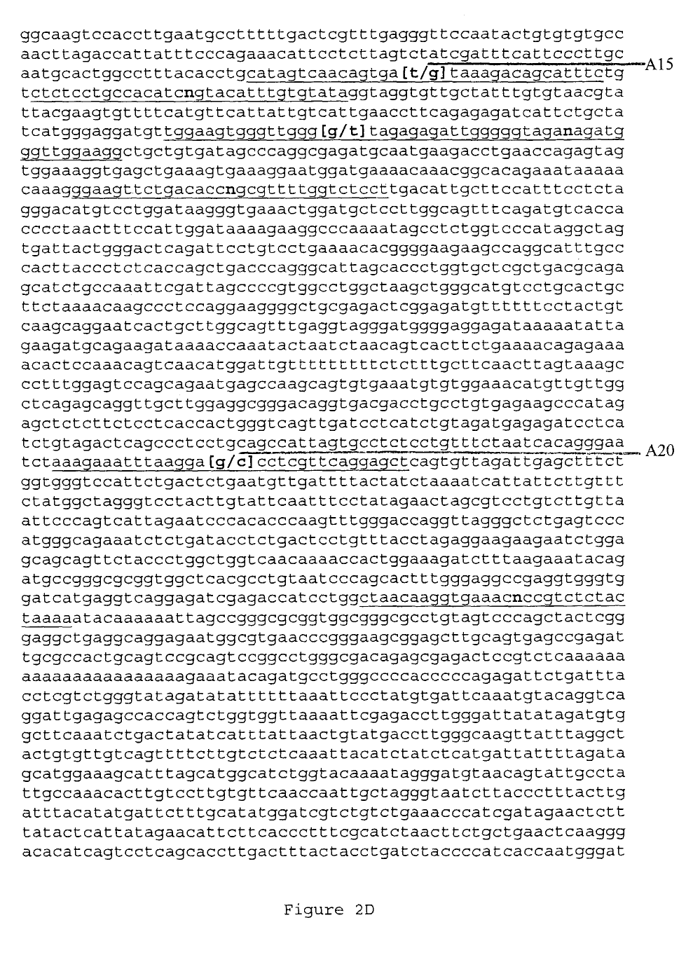

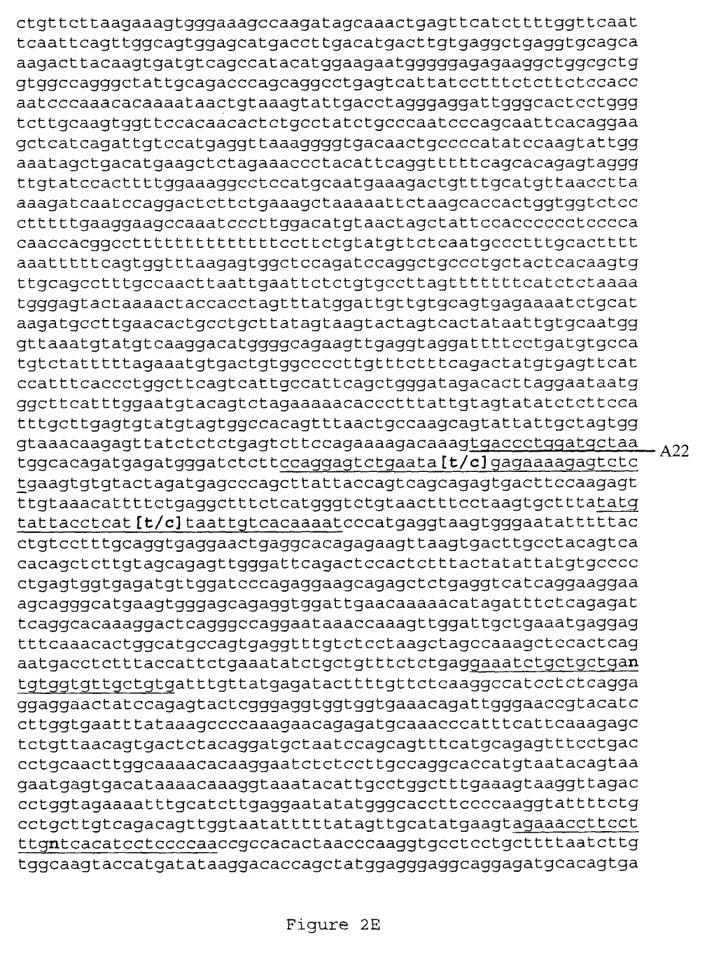

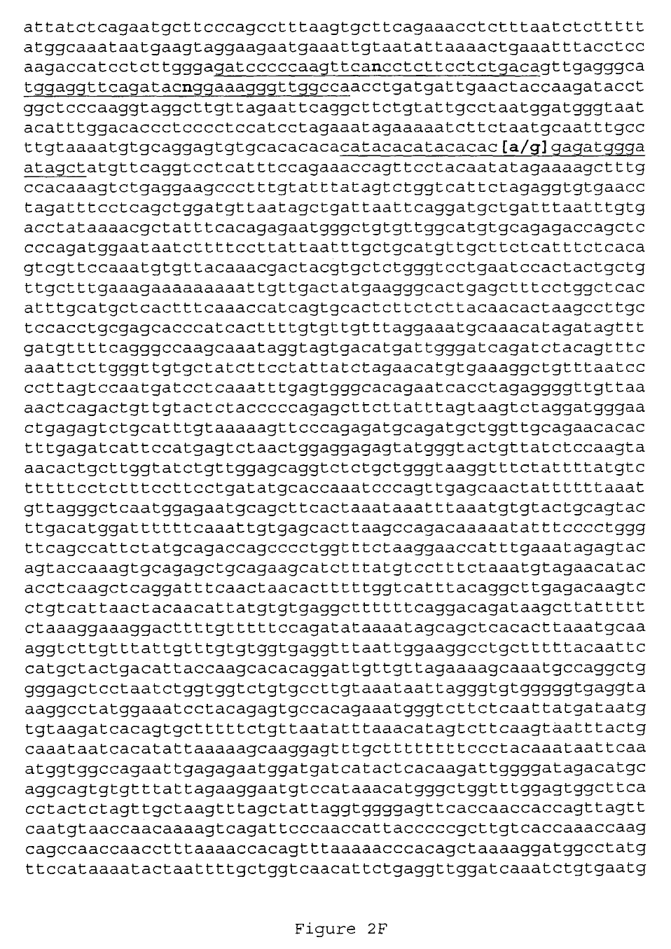

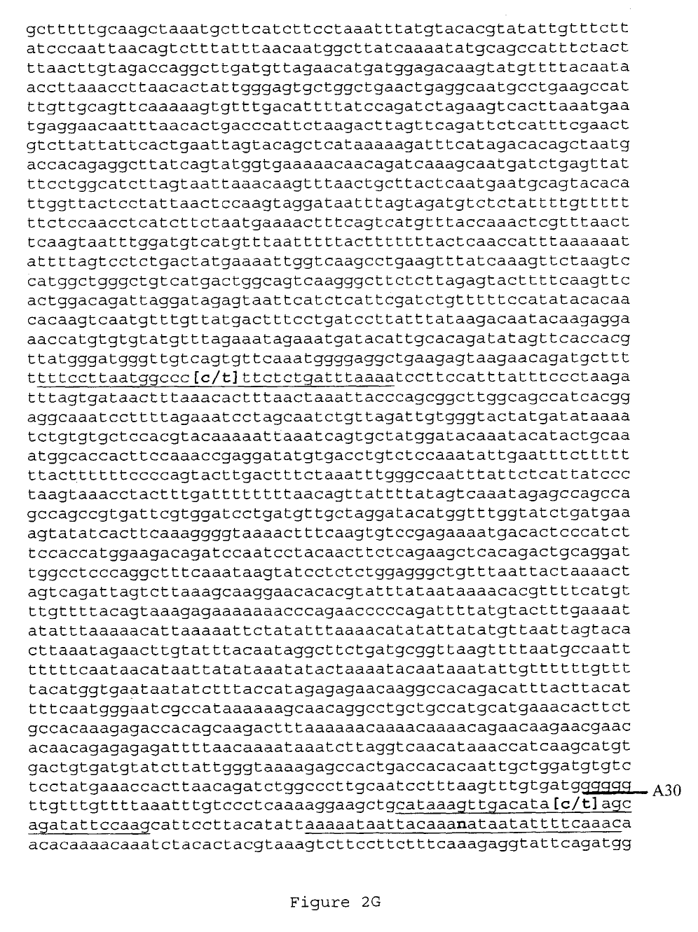

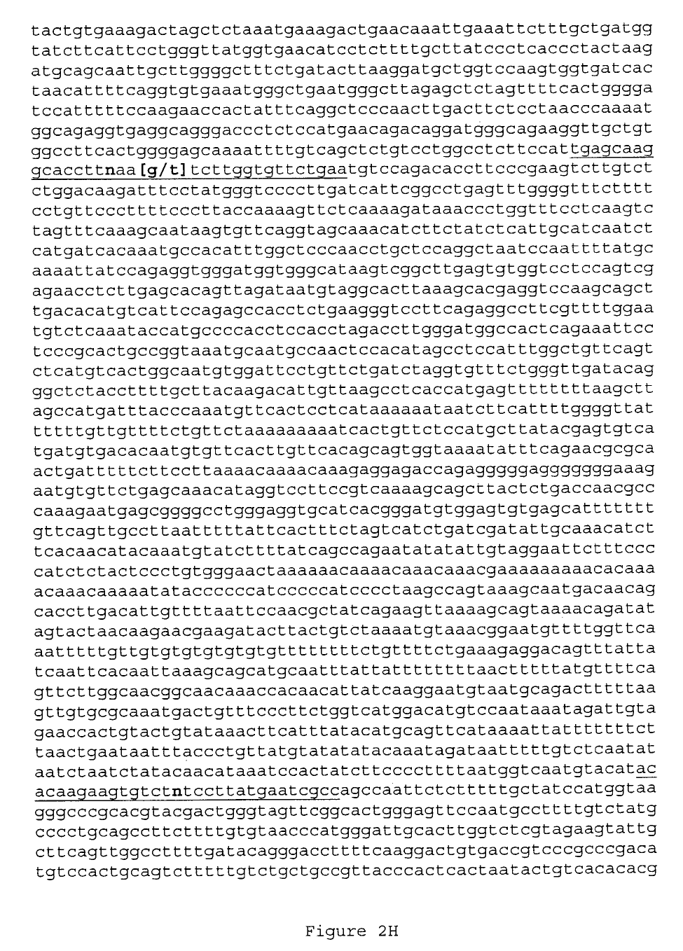

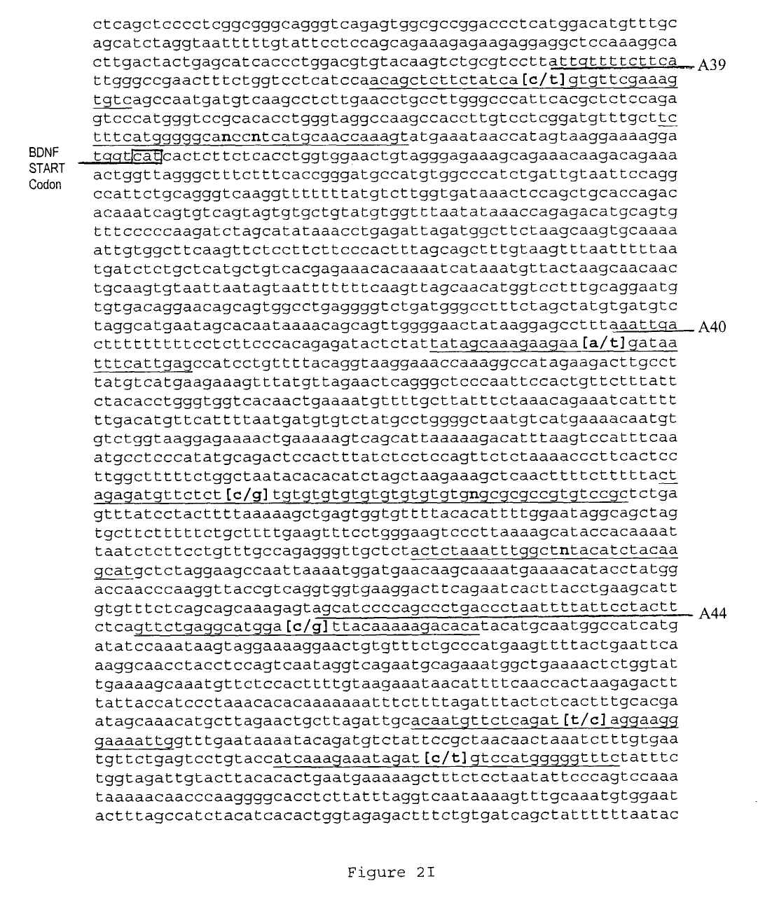

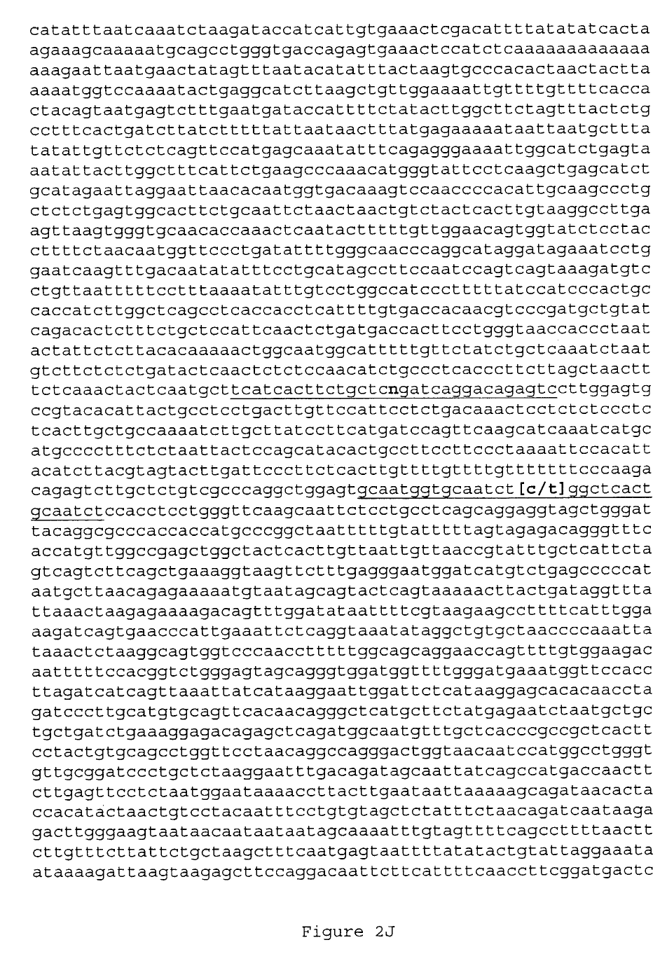

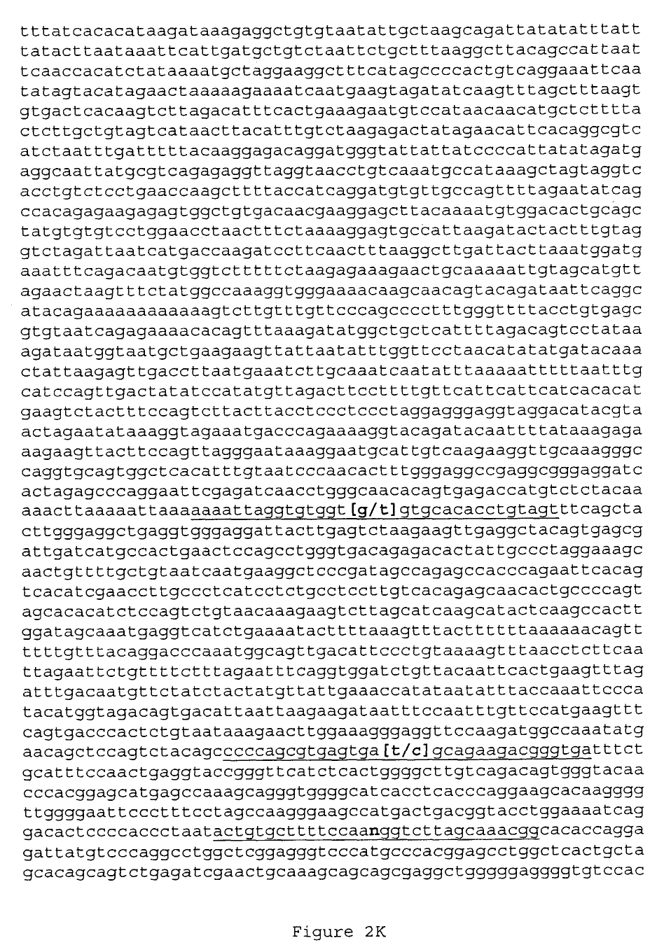

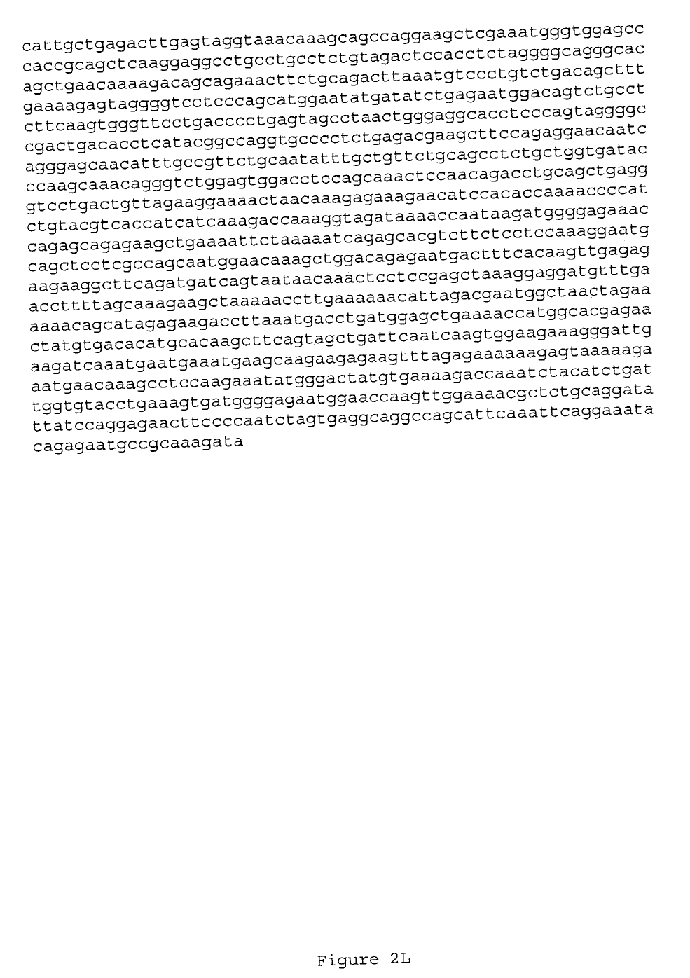

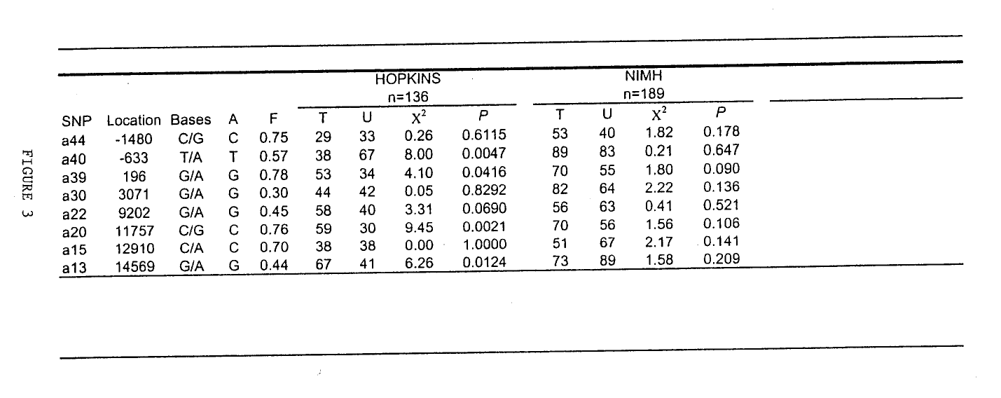

Twenty-three SNPs were chosen for genotyping based on allele frequency estimates from the resequencing. Genotyping was successful for 10 (2 have a rare-minor allele frequency≦1%, 4 are monomorphic in the patient samples, and 9 failed in genotyping format). The data for the 8 informative SNPs are presented in FIG. 3, and their relative positions are indicated in FIG. 2.

As described herein, it has been discovered that polymorphisms in the gene for BDNF, or polymorphisms in linkage disequilibrium with such polymorphisms, form a haplotype that is correlated with incidence of neuropsychiatric disorders (e.g., bipolar disorder). In particular, it has been discovered that one or more predictive SNPs are correlated with a reduced incidence of bipolar disorder in the sample population assessed as described herein. In one embodiment, a predictive single nucleotide polymorphism from G to A at nucleotide position 196 (relative to the start codon of the BDNF gene as shown in FIG. 2), or at a nucleotide position corresponding thereto, resulting in an amino acid change from valine to methionine at amino acid position −63 (relative to the start of the mature protein), or at an amino acid position corresponding thereto, is correlated with a reduced incidence of bipolar disorder in the sample population assessed as described herein. Although, this polymorphism resides within the 132 amino acid precursor portion (the prepro portion) which is cleaved from the mature protein, it is in a region highly conserved across species.

As used herein, the phrase “at a nucleotide/amino acid position corresponding thereto” are intended to include equivalent sites (e.g., positions or residues) in BDNF genes from other species. For example, BDNF genes and flanking nucleotide sequence can be found, for example, in primates, mice and/or dogs. The corresponding positions can be identified, for example, by aligning the nucleotide or amino acid sequences of BDNF genes, as known to one of skill in the art, with SEQ ID NO: 2 and identifying the nucleotide position which is aligned with, e.g., nucleotide position 196 relative to the start codon of the BDNF gene.

Furthermore, data has shown that there is a variation from random (i.e., that which would be expected by chance) in the transmission of the reference (G) and variant (A) alleles from a parent who is heterozygous for the BDNF alleles to an offspring diagnosed with bipolar disorder. As seen in FIG. 3, the variant allele (A) is transmitted less frequently to the bipolar offspring than would be expected by chance, while the reference allele (G) is transmitted more frequently than would be expected by chance (T/U=53/34, P=0.0416). Thus, the SNP at nucleotide position 196 (also referred to as a39) is considered a predictive marker for bipolar disorder.

In another embodiment, a predictive single nucleotide polymorphism from T to A at nucleotide position −633 (relative to the start codon for BDNF; also referred to as a40) in FIG. 2, or at a nucleotide position corresponding thereto, has been discovered. Data has shown that there is a variation from random in the transmission of the reference (T) and variant (A) alleles from a parent who is heterozygous for the BDNF alleles to an offspring diagnosed with bipolar disorder. As seen in FIG. 3, the reference allele (T) is transmitted less frequently to the bipolar offspring than would be expected by chance, while the variant allele (A) is transmitted more frequently than would be expected by chance (T/U=38/67, P=0.0047).

In another embodiment, a predictive single nucleotide polymorphism from C to G at nucleotide position 11757 (relative to the start codon for BDNF; also referred to as a20) in FIG. 2, or at a nucleotide position corresponding thereto, has been discovered. Data has shown that there is a variation from random in the transmission of the reference (C) and variant (G) alleles from a parent who is heterozygous for the BDNF alleles to an offspring diagnosed with bipolar disorder. As seen in FIG. 3, the variant allele (G) is transmitted less frequently to the bipolar offspring than would be expected by chance, while the reference allele (C) is transmitted more frequently than would be expected by chance (T/U=59/30, P=0.0021).

In another embodiment, a predictive single nucleotide polymorphism from G to A at nucleotide position 14569 (relative to the start codon for BDNF; also referred to as a13) in FIG. 2, or at a nucleotide position corresponding thereto, has been discovered. Data has shown that there is a variation from random in the transmission of the reference (G) and variant (A) alleles from a parent who is heterozygous for the BDNF alleles to an offspring diagnosed with bipolar disorder. As seen in FIG. 3, the variant allele (A) is transmitted less frequently to the bipolar offspring than would be expected by chance, while the reference allele (G) is transmitted more frequently than would be expected by chance (T/U=67/41, P=0.0124).

Also as described herein, additional polymorphisms in linkage disequilibrium with the predictive SNPs described above have been discovered. In one embodiment, a SNP from C to G at nucleotide position −1480 (relative to the start codon for BDNF, also referred to as a44) in FIG. 2, or at a nucleotide position corresponding thereto, has been discovered. In another embodiment, a SNP from G to A at nucleotide position 3071 (relative to the start codon for BDNF; also referred to as a30) in FIG. 2, or at a nucleotide position corresponding thereto, has been discovered. In another embodiment, a SNP from G to A at nucleotide position 9202 (relative to the start codon for BDNF; also referred to as a22) in FIG. 2, or at a nucleotide position corresponding thereto, has been discovered. In another embodiment, a SNP from C to A at nucleotide position 12910 (relative to the start codon for BDNF; also referred to as a15) in FIG. 2, or at a nucleotide position corresponding thereto, has been discovered.

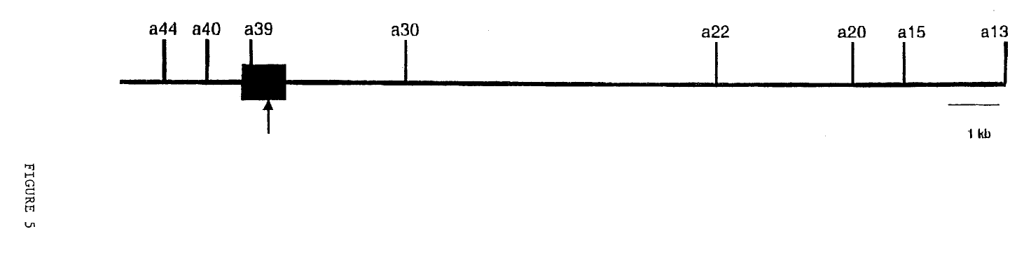

Strong linkage disequilibrium was observed between all 8 SNPS (i.e., the four predictive SNPs and the four uncertain SNPs described above). The extent of LD between adjacent SNPs was determined by calculating the statistic D′. An absolute value of D′ of 1 indicates complete LD, while 0 corresponds to no LD. All pairwise combinations of SNPs tested across this region in both the Hopkins and NIMH dataset are in nearly complete LD (Hopkins D′≧0.90; NIMH D′≧0.88). FIG. 5 depicts the location of the 8 SNPs over seventeen kb of genomic sequence.

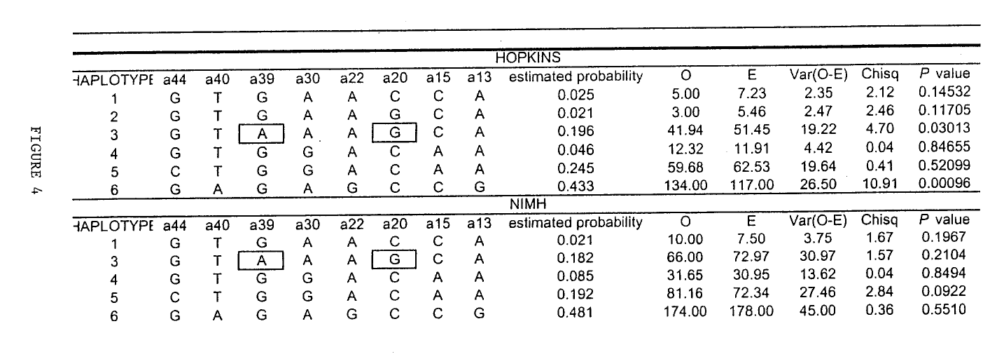

Moreover, multiple haplotype analysis performed as described herein reveals that 6 haplotypes, each with a probability of greater than 2%, account for 96.5% of the haplotype diversity (FIG. 4). Haplotype 6 is significantly overtransmitted to the bipolar probands, and haplotype 3 is undertransmitted in the Hopkins data set.

Thus, it appears that there exist haplotypes formed by the four predictive SNPs and the four uncertain SNPs described above that are associated with the BDNF gene and which contribute to the presence, absence, or severity of neuropsychiatric disorder (e.g., bipolar disorder).

Thus, the invention relates to a method for predicting the likelihood that an individual will have a neuropsychiatric disorder, or for aiding in the diagnosis of a neuropsychiatric disorder, or predicting the likelihood of having reduced symptomology associated with a neuropsychiatric disorder, comprising the steps of obtaining a DNA sample from an individual to be assessed and determining the nucleotide present at one or more of nucleotide positions −633, 196, 11757 and 14569 relative to the start codon for the BDNF gene. In a preferred embodiment, the nucleotides present at one or more of these nucleotide positions are determined, in a particularly preferred embodiment the nucleotides present at two or more of these nucleotide positions are determined, in a more particularly preferred embodiment the nucleotides present at three or more of these nucleotide positions are determined, and in an even more particularly preferred embodiment the nucleotides present at four or more of these nucleotide positions are determined.

In one embodiment the BDNF gene has the nucleotide sequence of SEQ ID NO: 1. The presence of one or more of an adenine (the reference nucleotide) at the predictive marker position −633, a guanine (the reference nucleotide) at the predictive marker position 196, a cytosine (the reference nucleotide) at the predictive marker position 11757, or a guanine (the reference nucleotide) at the predictive marker position 14569, indicates that the individual has a greater likelihood of having a neuropsychiatric disorder, or a greater likelihood of having severe symptomology associated with a neuropsychiatric disorders, than if that individual had the variant nucleotide at one or more of these positions. Conversely, the presence of one or more of a thymine (the variant nucleotide) at the predictive marker position −633, an adenine (the variant nucleotide) at the predictive marker position 196, a guanine (the variant nucleotide) at the predictive marker position 11757, or an adenine (the variant nucleotide) at the predictive marker position 14569 indicates that the individual has a reduced likelihood of having a neuropsychiatric disorder or a likelihood of having reduced symptomology associated with a neuropsychiatric disorder than if that individual had the reference nucleotide at one or more of these positions.

In a particular embodiment, the individual is an individual at risk for development of a neuropsychiatric disorder. In another embodiment the individual exhibits clinical symptomology associated with a neuropsychiatric disorder. In one embodiment, the individual has been clinically diagnosed as having a neuropsychiatric disorder. In a preferred embodiment, the neuropsychiatric disorder is bipolar disorder.

The genetic material to be assessed can be obtained from any nucleated cell from the individual. For assay of genomic DNA, virtually any biological sample (other than pure red blood cells) is suitable. For example, convenient tissue samples include whole blood, semen, saliva, tears, urine, fecal material, sweat, skin and hair. For assay of cDNA or mRNA, the tissue sample must be obtained from an organ in which the target nucleic acid is expressed. For example, cells from the central nervous system (such as cells of the hippocampus), heart, brain, lung and skeletal muscle are suitable sources for obtaining cDNA for the BDNF gene.

Many of the methods described herein require amplification of DNA from target samples. This can be accomplished by e.g., PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202.

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The nucleotides which occupy the polymorphic sites of interest (e.g., nucleotide positions −1480, −633, 196, 3071, 9202, 11757, 12910, and 14569 of BDNF) can be identified by a variety methods, such as Southern analysis of genomic DNA; direct mutation analysis by restriction enzyme digestion; Northern analysis of RNA; denaturing high pressure liquid chromatography (DHPLC); gene isolation and sequencing; hybridization of an allele-specific oligonucleotide with amplified gene products; single base extension (SBE); or analysis of the BDNF protein. A sampling of suitable procedures are discussed below.

1. Allele-Specific Probes

The design and use of allele-specific probes for analyzing polymorphisms is described by e.g., Saiki et al., Nature 324, 163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-3020 C., or equivalent conditions, are suitable for allele-specific probe hybridizations. Equivalent conditions can be determined by varying one or more of the parameters given as an example, as known in the art, while maintaining a similar degree of identity or similarity between the target nucleotide sequence and the primer or probe used.

Some probes are designed to hybridize to a segment of target DNA such that the polymorphic site aligns with a central position (e.g., in a 15-mer at the 7 position; in a 16-mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

2. Tiling Arrays

The polymorphisms can also be identified by hybridization to nucleic acid arrays, some examples of which are described in WO 95/11995. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

3. Allele-Specific Primers

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer which hybridizes at a distal site. Amplification proceeds from the two primers, resulting in a detectable product which indicates the particular allelic form is present. A control is usually performed with a second pair of primers, one of which shows a single base mismatch at the polymorphic site and the other of which exhibits perfect complementarity to a distal site. The singlebase mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

4. Direct-Sequencing

The direct analysis of the sequence of polymorphisms of the present invention can be accomplished using either the dideoxy chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

5. Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W. H. Freeman and Co, New York, 1992), Chapter 7.

6. Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

7. Single-Base Extension

An alternative method for identifying and analyzing polymorphisms is based on single-base extension (SBE) of a fluorescently-labeled primer coupled with fluorescence resonance energy transfer (FRET) between the label of the added base and the label of the primer. Typically, the method, such as that described by Chen et al., (PNAS 94:10756-61 (1997), incorporated herein by reference) uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently labeled dideoxyribonucleotides (ddNTPs) in dye-terminator sequencing fashion, except that no deoxyribonucleotides are present. An increase in fluorescence of the added ddNTP in response to excitation at the wavelength of the labeled primer is used to infer the identity of the added nucleotide.

More than one phenotypic trait may be affected by the SNPs described herein. For example, other neuropsychiatric disorders which are believed to be alternate expressions of a bipolar disorder, including schizoaffective disorder, recurrent unipolar depression and hypomania (bipolar II disorder), may also be affected by the BDNF polymorphisms described herein. Additionally, the described polymorphisms may predispose an individual to a distinct mutation that is causally related to a certain phenotype, such as susceptibility or resistance to bipolar disorder. The discovery of these polymorphisms and their correlation with bipolar disorder facilitates biochemical analysis of the variants and the development of assays to characterize the variants and to screen for pharmaceuticals that interact directly with one or another form of the protein.

Alternatively, a polymorphism may be one of a group of two or more polymorphisms in the BDNF gene, or in linkage disequilibrium with such polymorphisms, that form a haplotype which contributes to the presence, absence or severity of the neuropsychiatric disorder, e.g., bipolar disorder. An assessment of other polymorphisms within the BDNF gene, or in linkage disequilibrium with such polymorphisms, can be undertaken, and the separate and combined effects of these polymorphisms on the neuropsychiatric disorder phenotype can be assessed.

Correlation between a particular phenotype, e.g., the bipolar phenotype, and the presence or absence of a particular allele is performed for a population of individuals who have been tested for the presence or absence of the phenotype. Correlation can be performed by standard statistical methods such as a Chi-squared test and statistically significant correlations between polymorphic form(s) and phenotypic characteristics are noted. For example, as described herein, it has been found that the presence of the BDNF variant allele having an A at polymorphic site 196, correlates negatively with bipolar disorder with a p-value of 0.0416. Additionally, analysis of a haplotype formed by the eight SNPs disclosed herein (i.e., at positions −1480, −633, 196, 3071, 9202, 11757, 12910 and 14569) using TRANSMIT shows an association with bipolar disorder with a global p-value of 0.034.

This correlation can be exploited in several ways. In the case of a strong correlation, detection of the polymorphic form in an individual may justify immediate administration of treatment, or at least the institution of regular monitoring of the individual. Detection of a polymorphic form correlated with a disorder in a couple contemplating a family may also be valuable to the couple in their reproductive decisions. For example, the female partner might elect to undergo in vitro fertilization to avoid the possibility of transmitting such a polymorphism from her husband to her offspring. In the case of a weaker, but still statistically significant correlation between a polymorphic form and a particular disorder, immediate therapeutic intervention or monitoring may not be justified. Nevertheless, the individual can be motivated to begin simple life-style changes (e.g., therapy or counseling) that can be accomplished at little cost to the individual but confer potential benefits in reducing the risk of conditions to which the individual may have increased susceptibility by virtue of the particular allele. Furthermore, identification of a polymorphic form correlated with enhanced receptiveness to one of several treatment regimes for a disorder indicates that this treatment regime should be followed for the individual in question.

Furthermore, it may be possible to identify a physical linkage between a genetic locus associated with a trait of interest (e.g., bipolar disorder) and polymorphic markers that are not associated with the trait, but are in physical proximity with the genetic locus responsible for the trait and co-segregate with it. Such analysis is useful for mapping a genetic locus associated with a phenotypic trait to a chromosomal position, and thereby cloning gene(s) responsible for the trait. See Lander et al., Proc. Natl. Acad. Sci. (USA) 83, 7353-7357 (1986); Lander et al., Proc. Natl. Acad. Sci. (USA) 84, 2363-2367 (1987); Donis-Keller et al., Cell 51, 319-337 (1987); Lander et al., Genetics 121, 185-199 (1989)). Genes localized by linkage can be cloned by a process known as directional cloning. See Wainwright, Med. J. Australia 159, 170-174 (1993); Collins, Nature Genetics 1, 3-6 (1992).

Linkage studies are typically performed on members of a family. Available members of the family are characterized for the presence or absence of a phenotypic trait and for a set of polymorphic markers. The distribution of polymorphic markers in an informative meiosis is then analyzed to determine which polymorphic markers cosegregate with a phenotypic trait. See, e.g., Kerem et al., Science 245, 1073-1080 (1989); Monaco et al., Nature 316, 842 (1985); Yamoka et al., Neurology 40, 222-226 (1990); Rossiter et al., FASEB Journal 5, 21-27 (1991).

Linkage is analyzed by calculation of LOD (log of the odds) values. A lod value is the relative likelihood of obtaining observed segregation data for a marker and a genetic locus when the two are located at a recombination fraction θ, versus the situation in which the two are not linked, and thus segregating independently (Thompson & Thompson, Genetics in Medicine (5th ed, W. B. Saunders Company, Philadelphia, 1991); Strachan, “Mapping the human genome” in The Human Genome (BIOS Scientific Publishers Ltd, Oxford), Chapter 4). A series of likelihood ratios are calculated at various recombination fractions (θ), ranging from θ=0.0 (coincident loci) to θ=0.50 (unlinked). Thus, the likelihood at a given value of θ is: probability of data if loci linked at θ to probability of data if loci unlinked. The computed likelihoods are usually expressed as the log₁₀ of this ratio (i.e., a lod score). For example, a lod score of 3 indicates 1000:1 odds against an apparent observed linkage being a coincidence. The use of logarithms allows data collected from different families to be combined by simple addition. Computer programs are available for the calculation of lod scores for differing values of θ (e.g., LIPED, MLINK (Lathrop, Proc. Nat. Acad. Sci. (USA) 81, 3443-3446 (1984)). For any particular lod score, a recombination fraction may be determined from mathematical tables. See Smith et al., Mathematical tables for research workers in human genetics (Churchill, London, 1961); Smith, Ann. Hum. Genet. 32, 127-150 (1968). The value of θ at which the lod score is the highest is considered to be the best estimate of the recombination fraction.

Positive lod score values suggest that the two loci are linked, whereas negative values suggest that linkage is less likely (at that value of θ) than the possibility that the two loci are unlinked. By convention, a combined lod score of +3 or greater (equivalent to greater than 1000:1 odds in favor of linkage) is considered definitive evidence that two loci are linked. Similarly, by convention, a negative lod score of −2 or less is taken as definitive evidence against linkage of the two loci being compared. Negative linkage data are useful in excluding a chromosome or a segment thereof from consideration. The search focuses on the remaining non-excluded chromosomal locations.

In another embodiment, the invention relates to pharmaceutical compositions comprising a variant gene product. As used herein, a variant gene product is intended to mean gene products which are encoded by the variant allele of a gene and includes, but is not limited to, a full-length, or substantially full length, variant gene product, or biologically active fragments of the gene product, or analogs thereof. Variant gene products comprise at least one variant nucleotide at a polymorphic site disclosed herein. Biologically active fragments include any portion of the encoded full-length polypeptide which confers a biological function on the variant gene product, including ligand binding and antibody binding. Ligand binding includes binding by nucleic acids, proteins or polypeptides, small biologically active molecules, or large cellular structures.

A variant gene product is also intended to mean gene products which have altered expression levels or expression patterns which are caused, for example, by the variant allele of a regulatory sequence(s). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc.

A variant polypeptide or protein, or fragment thereof, of the present invention can be formulated with a physiologically acceptable medium to prepare a pharmaceutical composition. A variant polypeptide or protein is one which is encoded by a variant gene product and preferable comprises a variant amino acid as described herein. The particular physiological medium may include, but is not limited to, water, buffered saline, polyols (e.g., glycerol, propylene glycol, liquid polyethylene glycol) and dextrose solutions. The optimum concentration of the active ingredient(s) in the chosen medium can be determined empirically, according to procedures well known to medicinal chemists, and will depend on the ultimate pharmaceutical formulation desired. Methods of introduction of exogenous peptides at the site of treatment include, but are not limited to, intradermal, intramuscular, intraperitoneal, intravenous, subcutaneous, oral and intranasal. Other suitable methods of introduction can also include rechargeable or biodegradable devices and slow release polymeric devices. The pharmaceutical compositions of this invention can also be administered as part of a combinatorial therapy with other agents and treatment regimens.

Polyclonal and/or monoclonal antibodies that specifically bind to variant gene products (e.g., protein or polypeptide) but not to corresponding reference gene products are also provided. Antibodies can be made by injecting mice or other animals with the variant gene product or synthetic peptide fragments thereof comprising the variant portion. Monoclonal antibodies are screened as are described, for example, in Harlow & Lane, Antibodies, A Laboratory Manual, Cold Spring Harbor Press, New York (1988); Goding, Monoclonal antibodies, Principles and Practice (2d ed.) Academic Press, New York (1986). Monoclonal antibodies are tested for specific immunoreactivity with a variant gene product and lack of immunoreactivity to the corresponding prototypical gene product. These antibodies are useful in diagnostic assays for detection of the variant form, or as an active ingredient in a pharmaceutical composition.

The invention further pertains to compositions, e.g., vectors, comprising a nucleotide sequence encoding a variant gene product. For example, variant genes can be expressed in an expression vector in which a variant gene is operably linked to a native or other promoter. Usually, the promoter is a eukaryotic promoter for expression in a mammalian cell. The transcription regulation sequences typically include a heterologous promoter and optionally an enhancer which is recognized by the host. The selection of an appropriate promoter, for example trp, lac, phage promoters, glycolytic enzyme promoters and tRNA promoters, depends on the host selected. Commercially available expression vectors can be used. Vectors can include host-recognized replication systems, amplifiable genes, selectable markers, host sequences useful for insertion into the host genome, and the like.

The means of introducing the expression construct into a host cell varies depending upon the particular construction and the target host. Suitable means include fusion, conjugation, transfection, transduction, electroporation or injection, as described in Sambrook, supra. A wide variety of host cells can be employed for expression of the variant gene, both prokaryotic and eukaryotic. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the variant gene product to produce an appropriate mature polypeptide. Processing includes glycosylation, ubiquitination, disulfide bond formation, general post-translational modification, and the like.

It is also contemplated that cells can be engineered to express a variant allele of the invention by gene therapy methods. For example, DNA encoding a variant gene product, or an active fragment or derivative thereof, can be introduced into an expression vector, such as a viral vector, and the vector can be introduced into appropriate cells in an animal. In such a method, the cell population can be engineered to inducibly or constitutively express active variant gene product. In a preferred embodiment, the vector is delivered to the bone marrow, for example as described in Corey et al. (Science 244:1275-1281 (1989)).

The invention further relates to the use of compositions (i.e., agonists) which enhance or increase the level or activity of a variant gene product, or a functional portion thereof, or compositions which mimic the activity of a variant gene product, for use in the treatment of a phenotype. The invention also relates to the use of compositions (i.e., antagonists) which reduce or decrease the level or activity of a varient gene product, or a functional portion thereof, for use in the treatment of a phenotype.

The invention also relates to constructs which comprise a vector into which a sequence of the invention has been inserted in a sense or antisense orientation. For example, a vector comprising a nucleotide sequence which is antisense to a variant allele may be used as an antagonist of the activity of a reference allele. Alternatively, a vector comprising a nucleotide sequence of a variant allele may be used therapeutically to treat a phenotype. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses) that serve equivalent functions.

Preferred recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell).

The expression vectors of the invention can be introduced into host cells to thereby produce proteins or peptides, including fusion proteins or peptides, encoded by nucleic acids as described herein. The recombinant expression vectors of the invention can be designed for expression of a polypeptide of the invention in prokaryotic or eukaryotic cells, e.g., bacterial cells such as E. coli, insect cells (using baculovirus expression vectors), yeast cells or mammalian, e.g., human, cells. Suitable host cells are discussed further in Goeddel, supra. Alternatively, the recombinant expression vector can be transcribed and translated in vitro, for example using T7 promoter regulatory sequences and T7 polymerase.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a nucleic acid of the invention can be expressed in bacterial cells (e.g., E. coli), insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

A host cell of the invention, such as a prokaryotic or eukaryotic host cell in culture, can be used to produce (i.e., express) a polypeptide of the invention. Accordingly, the invention further provides methods for producing a polypeptide using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a nucleic acid of the invention have been introduced. Such host cells can then be used to create non-human transgenic animals in which exogenous nucleotide sequences have been introduced into their genome or homologous recombinant animals in which endogenous nucleotide sequences have been altered. Such animals are useful for studying the function and/or activity of the nucleotide sequence and for identifying and/or evaluating modulators of their activity. As used herein, a “transgenic animal” is a non-human animal, preferably a mammal, more preferably a rodent such as a rat or mouse, in which one or more of the cells of the animal includes the exogenous nucleotide sequence. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of a gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a nucleic acid of the invention into the male pronuclei of a fertilized oocyte, e.g., by microinjection, retroviral infection, and allowing the oocyte to develop in a pseudopregnant female foster animal. The sequence can be introduced as a transgene into the genome of a non-human animal. If the exogenous nucleotide sequence encodes a protein, intronic sequences and polyadenylation signals can also be included in the transgene to increase the efficiency of expression of the transgene. A tissue-specific regulatory sequence(s) can be operably linked to the transgene to direct expression of a polypeptide in particular cells. Methods for generating transgenic animals via embryo manipulation and microinjection, particularly animals such as mice, have become conventional in the art and are described, for example, in U.S. Pat. Nos. 4,736,866 and 4,870,009, 4,873,191 and in Hogan, Manipulating the Mouse Embryo (Cold Spring Harbor Laboratory Press, Cold Spring Harbor, N.Y., 1986). Similar methods are used for production of other transgenic animals. A transgenic founder animal can be identified based upon the presence of the exogenous nucleotide sequence in its genome and/or expression of mRNA in tissues or cells of the animals. A transgenic founder animal can then be used to breed additional animals carrying the transgene. Moreover, transgenic animals carrying a transgene comprising the exogenous nucleotide sequence can further be bred to other transgenic animals carrying other transgenes.

The invention also relates to the use of the variant and reference SNPs to guide efforts to identify the causative mutation for a phenotype or to identify or synthesize agents useful in the treatment of a phenotype. For example, amino acids that are essential for function can be identified by methods known in the art, such as site-directed mutagenesis or alanine-scanning mutagenesis (Cunningham et al., Science, 244:1081-1085 (1989)). The latter procedure introduces single alanine mutations at every residue in the molecule. The resulting mutant molecules are then tested for biological activity in vitro, or in vitro proliferative activity. Sites that are critical for polypeptide activity can also be determined by structural analysis such as crystallization, nuclear magnetic resonance or photoaffinity labeling (Smith et al., J. Mol. Biol., 224:899-904 (1992); de Vos et al. Science, 255:306-312 (1992)).

Another aspect of the invention pertains to monitoring the influence of agents (e.g., drugs, compounds) on the activity or function of SNPs of the invention in clinical trials. An exemplary method for detecting the presence or absence of proteins or nucleic acids of the invention in a biological sample involves obtaining a biological sample from a test subject and contacting the biological sample with a compound or an agent capable of detecting the protein, or nucleic acid (e.g., mRNA, genomic DNA), such that the presence of the protein or nucleic acid is detected in the biological sample. A preferred agent for detecting mRNA or genomic DNA is a labeled nucleic acid probe capable of hybridizing to mRNA or genomic DNA sequences described herein, preferably in an allele-specific manner. The nucleic acid probe can be, for example, a full-length nucleic acid, or a portion thereof, such as an oligonucleotide of at least 15, 30, 50, 100, 250 or 500 nucleotides in length and sufficient to specifically hybridize under stringent conditions to appropriate mRNA or genomic DNA. Other suitable probes for use in the diagnostic assays of the invention are described herein. Other agents for use in the diagnostic assays of the invention are antibodies described herein. These antibodies can be used to detect the presence or absence of the reference or variant gene product.

The invention also encompasses kits for detecting the presence of proteins or nucleic acid molecules of the invention in a biological sample. For example, the kit can comprise a labeled compound or agent (e.g., nucleic acid molecule, antibody, etc.) capable of detecting protein, DNA or MRNA in a biological sample; means for determining the amount of protein, DNA or mRNA in the sample; and means for comparing the amount of in the sample with a standard. The compound or agent can be packaged in a suitable container. The kit can further comprise instructions for using the kit to detect protein or nucleic acid. In a preferred embodiment the labeled compound or agent detects either the alternate or reference form of the protein, DNA or mRNA, but not both.

The invention further relates to an oligonucleotide microarray having immobilized thereon a plurality of oligonucleotide probes. For example, the microarray can contain one or more probes for the polymorphic sites listed in FIG. 2. The preparation of such oligonucleotide microarrays is well known in the art. In a preferred embodiment the oligonucleotide probes hybridize only to one polymorphic form of the gene products disclosed herein.

In one embodiment, labeled nucleic acid (e.g., mRNA, genomic DNA) can be hybridized to DNA microarrays. Genomic DNA and mRNA isolation from cells in culture is known in the art. (see Sambrook et al, Molecular Cloning: A Laboratory Manual, Cold Spring Harbor Laboratory Press; Ausubel, F. M., et al., Current Protocols in Molecular Biology, Greene Publishing Assoc. and Wiley-Interscience 1987, & Supp. 49, 2000, the teachings of which are incorporated herein by reference). The nucleic acid is then labeled by standard methods. (see above references). The nucleic acid can be labeled with any appropriate molecule, for example fluorophores, biotin, radioactive nucleotide, and dye. In a preferred embodiment, the nucleic acid is fluorescently labeled.

The DNA microarray can be any high-density oligonucleotide microarray, for example, GeneChip™ HU 6800 (Affymetrix, Santa Clara, Calif.). Hybridization of labeled MRNA to the DNA microarray is known to those of skill in the art (see Tamayo et al., 1999. Proc. Natl. Acad. Sci., USA, 96:2907-2912; Eisen et al, 1999. Methods Enzymol., 303:179-205).

Quantitation of gene expression profiles from the hybridization of labeled mRNA/DNA microarray is performed by scanning the microarrays to measure the amount of hybridization at each position on the microarray with an Affymetrix scanner (Affymetrix, Santa Clara, Calif.). For each stimulus, a time series of mRNA levels (C={C1,C2,C3, . . . Cn}) and a corresponding time series of mRNA levels (M={M1,M2,M3, . . . Mn}) in control medium in the same experiment as the stimulus is obtained. Quantitative data is then analyzed. Ci and Mi are defined as relative steadystate mRNA levels, where i refers to the ith timepoint and n to the total number of timepoints of the entire timecourse. μM and σM are defined as the mean and standard deviation of the control time course, respectively.

Alternatively, labeled RNA can be hybridized to a filter or other solid support containing target nucleic acids that comprise the polymorphic sites disclosed herein. Hybridization and wash conditions should be stringent enough to ensure specific binding between labeled RNA and target sequences. Stringent hybridization and wash conditions are known in the art (see Sambrook et al., Molecular Cloning: A Laboratory Manual, Cold Spring Harbor Laboratory Press; Ausubel, F. M., et al., Current Protocols in Molecular Biology, Greene Publishing Assoc. and Wiley-Interscience 1987, & Supp. 49, 2000). Quantitation of specific hybridization can be performed by any suitable method including scintillation counting and densitometry.

The teachings of U.S. Pat. No. 6,458,541 and the other references and websites cited herein are incorporated herein by reference in their entirety.

### EXAMPLES

Sample Populations

Pedigrees were ascertained for a sample population of 136 trios (Hopkins samples) from inpatient and outpatient clinics in Maryland and Iowa. Most of the families have been previously described. The ascertainment criteria were: a treated bipolar I proband, at least 2 affected first-degree relatives and unilineal transmission. Probands had the following diagnoses: 106 BP1, 26 BP2 and 4 schizoaffective-manic (SA-M). All BP2 and SAM probands had a BP1 sibling and both siblings were used in the analyses. Diagnoses were established using the Research Diagnostic Criteria (RDC) (Spitzer R. L., et al., Clinical Criteria for Psychiatric Diagnosis and DSM-III. AM J Psychiatry 132: 1187-1192 (1975)).

90 SNPs in 76 candidate genes in the Hopkins sample of 136 parent-proband trios were genotyped. The choice of SNPs for genotyping was governed by the following rationale. It was reasoned that missense SNPs, because they result in amino acid changes, are those that are most likely to affect function, and thus missense SNPs were focused on when available. If these SNPs are the causal mutations, then genotyping will directly identify an association with the underlying disease or phenotype. However, because blocks of the genome are consistently inherited together in a population (linkage disequilibrium), nearby linked SNPs can also reveal an association to the underlying causative SNP. In order to take advantage of possible linkage disequilibrium, common silent SNPs were genotyped in some genes, especially when missense SNPs were unavailable. Since SNPs found in regulatory regions have the potential to control the level of gene expression, some SNPs identified in the 5′ or 3′ untranslated regions were also genotyped.

Given the heterogeneous nature of bipolar disorder, it was anticipated that any effects that were likely to be found would be weak. In order to be efficient, and yet retain power to detect modest effects, a study was designed as follows: an initial set of trios was used as a screening sample; any nominally positive results were genotyped in additional, larger independent sample populations. This is analogous to performing a genome scan in a subset of patients and then following up only suggestive linkage peaks in a larger independent sample for confirmation.

In the initial screening set of SNPs, a valine to methionine polymorphism (V66M) in brain-derived neurotrophic factor (BDNF) displayed a nominal association to bipolar disorder (p<0.05). BDNF is synthesized from a larger precursor peptide. While the valine to methionine SNP is located in the portion of the molecule thought to be cleaved from the mature peptide, this region is conserved across many species. TDT analysis in parent-proband trios from the Hopkins sample revealed that the comrnon valine variant is transmitted significantly more often from parents to their bipolar probands than expected by chance (T/U=53/34, p=0.04).

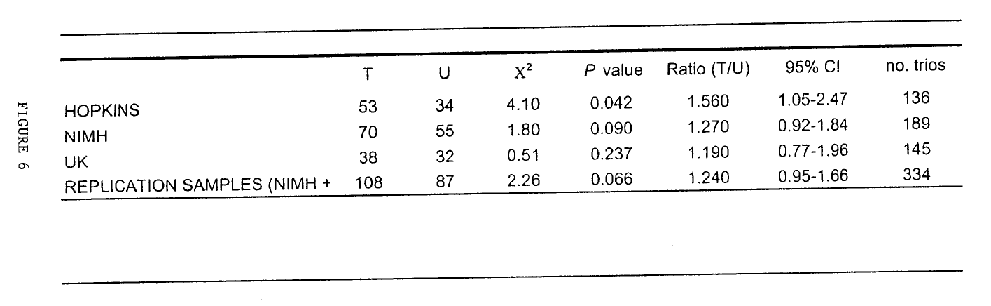

In an attempt to replicate the above data, a second sample of 189 trios obtained from the NIMH Genetics Initiative (NIMH sample), and a third sample of 145 trios obtained from the UK collaboration between the University of Wales College of Medicine in Cardiff and the University of Birmingham in Birmingham, were genotyped. In both datasets the excess transmission of the valine allele was as in the original dataset (FIG. 6). The combined dataset of both replication samples shows excess transmission of the valine allele (T/U=108/87, p=0.066). In a sample of 333 non-BP patients tested for the same SNP, no excess transmission was observed (T/U=71/78). This indicates that the observed association is not the result of transmission ratio distortion.

SNP Identification

SNPs were identified from genomic DNA as previously described.³⁴ For those genes where genomic sequence was not available, the gene was amplified from RNA in an ethnically diverse panel of lymphoblastoid cell lines obtained from the Coriell cell repository. Approximately 50% of genes specific to the neuropsychiatry project could not be amplified from this source of RNA. To amplify those genes, brain RNA was obtained from the Stanley Foundation. Poly A+RNA was purified using the Oligotex Direct mRNA kit (Qiagen, Valencia, Calif.). Complementary DNA was transcribed from the poly A+RNA using 200 ng RNA, random hexamers (750 ng) and SuperScriptII RT (Life Technologies, Carlsbad, Calif.) in a reaction volume of a 100 μl containing 5×Superscript buffer, 50 nmol dNTPs, and 1 μmol DTT. Each gene was divided into several primary transcripts of 1 kilobase. The primary transcripts were amplified in a volume of 10 μl. Each reaction contained 10×PCR bufferII, 30 nmol MgCl₂, 2 nmol dNTPs, 1U AmpliTaq Gold (Perkin-Elmer, Boston, Mass.), and 2.5 nmol transcript specific primers. PCR conditions were as follows: 96° C.×10 min followed by 35 cycles of 96° C.×30 sec, 59° C.×30 sec and 72° C.×1 min. Each primary transcript was subdivided into overlapping 500 base pair fragments that were separately amplified in a volume of 18 μl containing 10×PCR buffer II, 41.25 nmol MgCl₂, 3 nmol dNTP's, 1.5U AmpliTaq Gold and 3.5 nmol fragment specific primers and amplified using the same PCR conditions. PCR products were prepared for sequencing using solid-phase reversible immobilization (SPRI) using Bangs Estapor SuperParamagnetic Microspheres (Bangs Laboratories, Inc., IN and Seradyn Uniform Microparticles, MN) as described. (see DeAngelis M. M. et al., Solid-Phase Reversible Immobilization for the Isolation of PCR Products. Nucleic Acids Res 23: 4742-4743 (1995)). Sequencing was performed using BigDye Terminator Chemistry (Perkin-Elmer, Boston, Mass.) on a capillary ABI 3700. SNP detection was as described previously. (see Cargill M., et al., Characterization of Single-Nucleotide Polymorphisms in Coding Regions of Human Genes. Nat Genet 22: 231-238 (1999)). Each fragment was sequenced in 32 individual DNA samples.

BDNF Resequencing

29 kb of contiguous genomic sequence was resequenced from genomic DNA as described. (see Cargikk M. et al., Characterization of Single-Nucleotide Polymorphisms in Coding Regions of Human Genes. Nat Genet 22: 231-238 (1999)). Sequence was obtained from the Human Genome Browser from human BAC AC068488.2_{—}13. Six patients with BP1 and two control DNAs obtained from the Coriell Cell Repository were resequenced. The bipolar patients used for resequencing were not included in the association analyses. Location of SNPs are marked with respect to the ATG of BDNF.

Genotyping Methodologies

Genotyping was performed by either single-base extension with fluorescent resonance energy transfer (SBE-FRET) (see Chen X. et al., Fluorescence Energy Transfer Detection as a Homogenous DNA Diagnostic Method. Proc Natl Acad Sci USA 94: 10756-10761 (1997)), single-base extension with fluorescence polarization (SBE-FP) (see Chen X. and Kwok P. Y. Template-Directed Dye-Terminator Incorporation (TDI) Assau: A Homogoneous DNA Diagnostic Method Based on Fluorescence Resonance Energy Transfer. Nucleic Acids Res 25: 347-353 (1997)) using a modified protocol as described previously (Altshuler D. et al. The Common PPARgamma Pro12Ala Polymorphism is Associated With Decreased Risk of Type 2 Diabetes. Nat Genet 26: 76-80 (2000)), or length-multiplexed single-base extension (LM-SBE) (see Lindblad-Toh K. et al., Large-Scale Discovery and Genotyping of Single-Nucleotide Polymorphisms in the Mouse. Nat Genet 24: 381-386 (2000)) with the following modifications. From sixteen to eighteen individual PCR amplicons were multiplexed rather than 50 amplicons. Secondary amplification for biotinylation of the PCR products was not performed. Rather, a single amplification was performed using both sequence-specific and universal biotinylation primers simultaneously. The PCR reaction contained AmpliTAQ Gold (4U, Perkin Elmer), dNTPs (0.5 mM), MgCl₂ (4 mM), genomic DNA (5 ng), specific locus primer mix (0.1 μM final concentration of each primer), biotinylated-T3 and biotinylated-T7 (0.125 μM) in the supplied buffer in a final volume of 20 μl using the following PCR conditions (95° C.×9 min, 37 cycles of 94° C.×30 sec, 55° C.×30 sec, and 72° C.×30 sec followed by a final extention of 72° C.×5 min). Primers used for genotyping can be found on the world-wide web at genome-wi.mit.edu. Genotyping of SNPs in BDNF was performed by mass spectrometry as follows. Primers were designed using SpectroDESIGNER software (Sequenom, Calif.) to have a Tm between 56 and 60 degrees with a mass range between 5000 and 8000 Da as described. (see Buetow K. H. et al., High-Throughput Development and Characterization of a Genomewide Collection of Gene-Based Single Nucleotide Polymorphism Markers By Chip-Based Matrix-Assisted Laser Desorption/Ionization Time-of-Flight Mass Spectrometry. Proc Natl Acad Sci USA 98: 581-584 (2001)). PCR amplification was performed as follows. Each reaction contained AmpliTAQ Gold (0.1U, Perkin Elmer), dNTPs (0.2 mM), MgCl₂ (1.5 mM), genomic DNA (5 ng), locus specific primers (0.2 μM final concentration of each primer), in the supplied buffer in a final volume of 6 μl using the following PCR conditions (92° C.×9 min, 46 cycles of 94° C.×20 sec, 56° C.×30 sec, and 72° C.×30 sec followed by a final extention of 72° C.×3 min). Following the PCR reaction dNTPs are removed by shrimp alkaline phosphatase (SAP) by adding 2 μl of SAP (0.3U) in Thermosequenase buffer and incubating at 37° C.×20 min, followed by inactivation at 85° C.×5 min. The homogeneous MassEXTEND reaction is performed by adding to the SAP-treated product 2 μl of a solution containing ddNTPs (0.50 μM each), dNTPs (0.50 μM each), MassEXTEND primers (0.6 nM), Thermosequenase buffer (Pharmacia, Peapack, N.J.), and Thermosequenase (0.063U/μl). The termination mix of ddNTPs and dNTPs is predicted by the SpectroDESIGNER software and is specific for each SNP genotyped. The reaction is thermocycled under the following conditions: 94° C.×2min, 40 cycles of 94° C.×5 sec, 40° C.×5 sec, 72° C.×5 sec, then 72° C.×5 min. SpectroCLEAN, a proprietary ion-exchange resin, is added to remove salt. The sample plate is rotated for 4 min at RT and then centrifuged for 1 min at 1400 rpm. Using a 24-pin SpectroPOINT, 7 nl of each reaction was then loaded onto each position of a 384-well SpectroCHIP preloaded with 7 nl of matrix (3-hydroxypicolinic acid). SpectroCHIPs were analyzed in automated mode by a MassARRAY RT mass spectrometer (Bruker-Sequenom) (see Buetow K. H. et al. High-Throughput Development and Characterization of a Genomewide Collection of Gene-Based Single Nucleotide Polymorphism Markers By Chip-Based Matrix-Assisted Laser Desorption/Ionization Time-of-Flight Mass Spectrometry. Proc Natl Acad Sci USA 98: 581-584 (2001)). The resulting spectra were analyzed by SPECTROTYPER software (Sequenom) after baseline correction and peak identification. DNA samples for which the SPECTROTYPER software could not define a genotype were subjected to clustering analysis by plotting the signal to noise ratio of the genotype-known and genotype-undetermined samples for each SNP. If the undetermined genotypes fell within clusters of known genotypes, genotypes were then obtained. The minimum acceptable signal to noise ratio was 5:1. Clusters were verified by two independent observers.

Statistical Analysis

Genotyping data was assessed in the following manner. SNPs were used for TDT analysis only if they met the following criteria: 1) greater than 90% of attempted genotypes were successful, 2) parental alleles were in Hardy-Weinberg equilibrium, and 3) zero or one Mendelian inheritance error was detected. Several methods of genotyping were used for this study. For those SNPs genotyped by more than one method (23%), a consensus genotype was obtained and used for TDT analysis. Significance of single and two-marker haplotypes were analyzed using the Perm 1 and Perm2 options in Genehunter2.0. Global analysis of multimarker haplotypes was performed using TRANSMIT v2.5.2 (see Clayton D. A Generalization of the Transmission/Disequilibrium Test for Uncertain-Haplotype Transmission. Am J Hum Genet 65: 1170-1177 (1999)). For these analyses the minimum haplotype frequency was set at 2%.

Multiple haplotype analysis was performed using TRANSMIT v2.5.2 and results are shown in FIG. 4. The program estimates the association from probabilities of a haplotype transmission to affected offspring even when there are uncertain marker haplotype assignments. In the Hopkins dataset, 13 haplotypes were observed, of these only 6 were present with probabilities greater than 2% and these accounted for the vast majority of the haplotype diversity (96.5%). The global P value for these 6 haplotypes is 0.034 (chisq=13.6, 6 df). There are three common major haplotypes, 3, 5 and 6 with rarer haplotypes 2 and 4 differing by only a single marker from 3 and 5 respectively. Of these, haplotype 6 is significantly overtransmitted to the bipolar probands and haplotype 3 is undertransmitted. In the NIMH dataset the global P value is not significant (chisq=5.3, 5 df). Furthermore, the overtransmission of haplotype 6 is not observed, while haplotype 5 shows some excess transmission and haplotype 3 is undertransmitted. Of note, both datasets share undertransmission of haplotype 3 which is uniquely marked by the rare A allele of the originally positive SNP, a39, and by a newly identified SNP, a20, that shows a more significant association to the phenotype in the Hopkins samples (T/U=59/30, P=0.0021). BDNF has limited haplotype diversity in both samples which is nearly fully characterized by the SNPs genotyped. A single undertransmitted haplotype is shared by these samples characterized by alleles of SNPs a39 and a20 that may mark a protective haplotype for bipolar disorder.

These 8 SNPs were also genotyped in a second sample of 189 trios from the NIMH Genetics Initiative (NIMH) in which the proband had BP1 disorder (n=176, 93%) or BP2 disorder (n=13, 7%). No individual SNPs were significant at the P=0.05 level. Although not statistically significant, two markers revealed excess transmission of a single allele (a39 and a20) in the direction observed in the original Hopkins dataset. Transmission disequilibrium was calculated for pairwise markers with 2 of 7 comparisons with P values<0.05. However, the overtransmitted haplotypes in the NIMH dataset differed from those observed in the Hopkins set. Because of the obvious difference between the percentages of BP2 in the two datasets, a single subanalysis of the BP1 and SAB samples together was performed to evaluate the role that phenotypic differences between the samples might play. Despite the decrease in sample size, the significance of the observation in the Hopkins trios improves slightly, while no statistically significant improvement is detected in the NIMH samples, or change in 2 marker haplotypes was observed.

To reconstruct accurate multi-marker haplotypes, a screen was performed to catch genotyping problems so that markers used for further analyses are in Hardy-Weinberg equilibrium (P<0.05) and have no Mendelian inheritance errors. Genotypes were obtained for an average of 96% of DNA samples tested. One test sample, the Hopkins sample, includes 109 trios where the proband has BP1 or SAB disorder (80%) and 27 trios where the proband has bipolar II (BP2) (20%). In this sample, an allele of four of the eight common SNPs tested was associated with transmission to bipolar patients (FIG. 3). Transmission disequilibrium was calculated for pairwise haplotypes for adjacent markers using the TDT2 implementation in GENEHUNTER2.0. All adjacent pairwise markers displayed significant transmission of one allele to the probands with chi-squares between 8 and 12. To assess the significance of these results, permutation tests were performed in which the genotype data was held constant but the transmission status of each chromosome (transmitted vs. untransmitted) was assigned at random. In one hundred thousand permutations of the entire data set of 8 markers, a single-allele chi-square value>9.45 was observed only 1416 times (corresponding to a gene-wide empirical p-value<0.01) and only 1135 simulations had 2 markers with chi-squares>11.52 (corresponding to a gene-wide empirical p-value<0.001). Thus, 4 markers within a 17 kb region associated with bipolar disorder were identified.

Probands, from the NIMH sample, had the following diagnoses: 149 BP1, 1 BP2 and 5 SA-M. Diagnoses of BP1 and SAM were established using DSMIII-R criteria and BP2 by the Research Diagnostic Criteria (RDC).

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for predicting the likelihood that an individual will have a neuropsychiatric disorder, comprising the steps of:
a) obtaining a nucleic acid sample from an individual to be assessed; and
b) determining the nucleotide present at nucleotide position 11,757 relative to the start codon of the brain-derived neurotrophic factor (BDNF) gene,
wherein the presence of a cytosine at position 11,757 indicates that the individual has a greater likelihood of having a neuropsychiatric disorder than an individual having a guanine at that position.

2. A method according to claim 1, wherein the neuropsychiatric disorder is bipolar disorder.

3. A method according to claim 1, wherein the individual is an individual at risk for development of bipolar disorder.

4. A method for predicting the likelihood that an individual will have a neuropsychiatric disorder, comprising the steps of:
a) obtaining a nucleic acid sample from an individual to be assessed; and
b) determining the nucleotide present at nucleotide position 14,569 relative to the start codon of the brain-derived neurotrophic factor (BDNF) gene,
wherein the presence of a guanine at position 14,569 indicates that the individual has a greater likelihood of having a neuropsychiatric disorder than an individual having an adenine at that position.

5. A method according to claim 4, wherein the neuropsychiatric disorder is bipolar disorder.

6. A method according to claim 4, wherein the individual is an individual at risk for development of bipolar disorder.

7. A method for predicting the likelihood that an individual will have reduced symptomology associated with a neuropsychiatric disorder, comprising the steps of:
a) obtaining a nucleic acid sample from an individual to be assessed; and
b) determining the nucleotide present at nucleotide position 11,757 relative to the start codon of the brain-derived neurotrophic factor (BDNF) gene,
wherein the presence of a guanine at position 11,757 indicates that the individual has a greater likelihood of having reduced symptomology associated with a neuropsychiatric disorder than an individual having a cytosine at that position.

8. A method according to claim 7, wherein the neuropsychiatric disorder is bipolar disorder.

9. A method for predicting the likelihood that an individual will have reduced symptomology associated with a neuropsychiatric disorder, comprising the steps of:
a) obtaining a nucleic acid sample from an individual to be assessed; and
b) determining the nucleotide present at nucleotide position 14,569 relative to the start codon of the brain-derived neurotrophic factor (BDNF) gene,
wherein the presence of an adenine at position 14,569 indicates that the individual has a greater likelihood of having reduced symptomology associated with a neuropsychiatric disorder than an individual having a guanine at that position.

10. A method according to claim 9, wherein the neuropsychiatric disorder is bipolar disorder.

11. A method of diagnosing or aiding in the diagnosis of a neuropsychiatric disorder in an individual comprising
a) obtaining a nucleic acid sample from the individual; and
b) determining the nucleotide present at two or more of nucleotide positions −633, 196, 11757 and 14569 relative to the start codon of the brain-derived neurotrophic factor (BDNF) gene,
wherein presence of one or more of an adenine at nucleotide position −633, a guanine at nucleotide position 196, a cytosine at nucleotide position 11757 or a guanine at nucleotide position 14569 is indicative of increased likelihood of a neuropsychiatric disorder in the individual as compared with an individual having one or more of a thymine at nucleotide position −633, an adenine at nucleotide position 196, a guanine at nucleotide position 11757 or an adenine at nucleotide position 14569.

12. The method of claim 11, wherein the nucleotide present at all four of nucleotide positions −633, 196, 11757 and 14569 relative to the start codon for the brain-derived neurotrophic factor (BDNF) gene is determined.

13. The method of claim 11, wherein the neuropsychiatric disorder is bipolar disorder.

14. A method of diagnosing or aiding in the diagnosis of a neuropsychiatric disorder in an individual comprising
a) obtaining a nucleic acid sample from the individual; and p1 b) determining the nucleotide present at two or more of nucleotide positions −633, 196, 11757 and 14569 relative to the start codon of the brain-derived neurotrophic factor (BDNF) gene,
wherein presence of one or more of a thymine at nucleotide position −633, an adenine at nucleotide position 196, a guanine at nucleotide position 11757 or an adenine at nucleotide position 14569 is indicative of decreased likelihood of a neuropsychiatric disorder in the individual as compared with an individual having one or more of a adenine at nucleotide position −633, a guanine at nucleotide position 196, a cytosine at nucleotide position 11757 or a guanine at nucleotide position 14569.

15. The method of claim 14, wherein the nucleotide present at all four of nucleotide positions −633, 196, 11757 and 14569 relative to the start codon for the brain-derived neurotrophic factor (BDNF) gene is determined.

16. The method according to claim 14, wherein the neuropsychiatric disorder is bipolar disorder.

17. A method for predicting the likelihood that an individual will have a neuropsychiatric disorder, comprising the steps of:
a) obtaining a nucleic acid sample from an individual to be assessed; and
b) determining the nucleotide present at two or more of nucleotide positions −633, 196, 11757 and 14569 relative to the start codon of the brain-derived neurotrophic factor (BDNF) gene,
wherein presence of one or more of an adenine at nucleotide position −633, a guanine at nucleotide position 196, a cytosine at nucleotide position 11757 or a guanine at nucleotide position 14569 is indicative of increased likelihood of a neuropsychiatric disorder in the individual as compared with an individual having one or more of a thymine at nucleotide position −633, an adenine at nucleotide position 196, a guanine at nucleotide position 11757 or an adenine at nucleotide position 14569.

18. The method according to claim 17, wherein the nucleotide present at all four of nucleotide positions −633, 196, 11757 and 14569 relative to the start codon for the brain-derived neurotrophic factor (BDNF) gene is determined.

19. The method according to claim 17, wherein the individual is an individual at risk for development of a neuropsychiatric disorder.

20. The method according to claim 17, wherein the neuropsychiatric disorder is bipolar disorder.

21. A nucleic acid molecule comprising:
the nucleic acid sequence of SEQ ID NO: 2, wherein the nucleic acid sequence comprises a polymorphic site at nucleotide positions −1480, −633, 3071, 9202, 11757, 12910 and 14569 relative to the start codon and wherein the nucleotide at one or more of the polymorphic sites is different from a nucleotide at a corresponding position in a corresponding reference allele.

22. The nucleic acid molecule according to claim 21, comprising one or more of a guanine at nucleotide position −1480, an adenine at nucleotide position −633, an adenine at nucleotide position 3071, an adenine at nucleotide position 9202, a guanine at nucleotide position 11757, an adenine at nucleotide position 12910 or an adenine at nucleotide position 14569.

23. An allele-specific oligonucleotide that hybridizes to the nucleic acid molecule of claim 21.

24. A nucleic acid molecule comprising:
a portion of the nucleic acid sequence of SEQ ID NO: 2, which is at least ten nucleotides in length and which comprises at least one of the polymorphic sites at nucleotide positions −1480, −633, 196, 3071, 9202, 11757, 12910 and 14569 relative to the start codon and wherein the nucleotide at one or more of the polymorphic sites is different from a nucleotide at a corresponding position in a corresponding reference allele.

25. An allele-specific oligonucleotide that hybridizes to the nucleic acid molecule of claim 24.

26. A method for predicting the likelihood of the presence of one or more nucleotides comprising a haplotype associated with a neuropsychiatric disorder, comprising the steps of:
a) obtaining a nucleic acid sample from an individual to be assessed; and
b) determining the nucleotide present at one or more of the nucleotide positions −1480, −633, 196, 3071, 9202, 11757, 12910 and 14569 relative to the start codon of the BDNF gene;
wherein the presence of one or more nucleotides comprising said haplotype at said nucleotide positions is indicative of the increased likelihood that one or more additional nucleotides comprising the haplotype will be present at one or more other of said nucleotide positions.

27. An oligonucleotide microarray having immobilized thereon a plurality of probes, wherein at least one of said probes is an allele-specific oligonucleotide which hybridizes specifically to a nucleic acid molecule comprising at least 10 contiguous nucleotides of the nucleic acid sequence of SEQ ID NO: 2 and comprising at least one of the polymorphic sites at nucleotide positions −1480, −633, 196, 3071, 9202, 11757, 12910 and 14569 relative to the start codon.
